(19) United States
(12) Patent Application Publication
(10) Pub. No.: US 2022/0310197 A1
(43) Pub. Date: Sep. 29, 2022

(54) SYSTEM AND METHOD FOR COMBATING PSEUDOMONAS AERUGINOSA AND STAPHYLOCOCCUS AUREUS INFECTIONS

(71) Applicant: Tata Consultancy Services Limited, Mumbai (IN)

(72) Inventors: Sharmila Shekhar MANDE, Pune (IN); Swadha ANAND, Pune (IN); Preethi Alagarai SAMPATH, Pune (IN)

(73) Assignee: Tata Consultancy Services Limited, Mumbai (IN)

(21) Appl. No.: 17/615,647

(22) PCT Filed: Jun. 4, 2020
(86) PCT No.: PCT/IB2020/055276

§ 371 Date: Dec. 1, 2021

(30) Foreign Application Priority Data
Jun. 6, 2019 (IN) 201921022525

(51) Int. Cl. G16B 15/00; C12N 15/10; G16B 30/10; G16B 20/00; G16B 40/00; C12Q 1/689; G16H 10/40

(52) U.S. Cl. CPC G16B 15/00; C12N 15/1065; G16B 30/10; G16B 20/00; G16B 40/00; C12Q 1/689; G16H 10/40; C12Q 2600/106; C12Q 1/6806

(57) ABSTRACT

Co-infection of Pseudomonas aeruginosa and Staphylococcus aureus, exacerbates the virulence gene expression as well as shows higher antibacterial resistance than when they cause infections individually thereby making the infection extremely difficult to combat. A method and system for combating infections due to Pseudomonas aeruginosa and Staphylococcus aureus has been provided. The system provides strategies to combat pathogenic infections caused by multi-drug resistant (MDR) and extensively drug resistant (XDR) strains of Pseudomonas aeruginosa and Staphylococcus aureus. The strategy involves identifying potential target sites, which can be utilized to compromise its multiple virulence or essential functions at the same time. The idea utilizes the fact that a conserved stretch of nucleotide sequence occurring multiple times on a pathogen genome encoding virulence factors or in vicinity of genes essential for pathogen survival encoded within the genome of the candidate pathogen can be targeted to disrupt the overall genetic machinery of the pathogen.

### PRIORITY CLAIM

This present application is a U.S. National stage Filing under 35 U.S.C. § 371 and claims priority from International Application No. PCT/IB2020/055276, filed on 4 Jun. 2020 which application claims priority under 35 U.S.C. § 119 from India Application No. 201921022525, filed on 6 Jun. 2019. The entire contents of the aforementioned application are incorporated herein by reference.

### TECHNICAL FIELD

The embodiments herein generally relate to the field of Pseudomonas aeruginosa and Staphylococcus aureus infections, and, more particularly, to a method and system for combating the problem of multidrug resistance resulting due to co-infection of Pseudomonas aeruginosa and Staphylococcus aureus.

### BACKGROUND

Infectious diseases caused by pathogenic bacteria pose a serious threat to the health sector across the world. Further, nosocomial or the hospital acquired infections (HAIs) are the fourth leading cause of diseases in industrialized countries. They are notoriously difficult to treat as the HAI agents develop resistance to most form of antibiotics. Two of the most difficult pathogens to treat among them are Pseudomonas aeruginosa and Staphylococcus aureus. Studies have shown that co-infection of these two pathogens, exacerbates the virulence gene expression as well as shows higher antibacterial resistance than when they cause infections individually thereby making the infection extremely difficult to treat.

These bacteria are predominantly treated with antibiotics and the rampant use of these has led to development of antibiotic resistance in most pathogens. These antibiotic resistance genes are further transferred between different bacteria utilizing several transfer methods. Additional problems arise which pertain to formation of biofilms in these bacteria which allows them to evade antibiotics. Several studies have shown that biofilm formation inhibitors (like several enzymes which degrade the matrix) as well as quorum quenchers (prevent biofilm formation) can prove useful in this regard. Despite utilizing these inhibitors several bacteria still escape the antibiotics and lead to relapse once the treatment is stopped.

In addition to that, immunological and antisense approaches has also been used. These treatments often lose their efficacy as bacteria often mutate the pathogenic factors used as targets thereby escaping the immune machinery of the host.

### SUMMARY

Embodiments of the present disclosure present technological improvements as solutions to one or more of the above-mentioned technical problems recognized by the inventors in conventional systems. For example, in one embodiment the system for combating infections due to Pseudomonas aeruginosa and Staphylococcus aureus is provided. The system comprises a sample collection module, a pathogen detection and DNA extraction module, a sequencer, one or more hardware processors, a memory, an administration module and an efficacy module. The sample collection module obtains a sample from an infected area. The pathogen detection and DNA extraction module isolates DNA/RNA from the obtained sample using one of a laboratory methods. The memory is in communication with the one or more hardware processors, wherein the one or more first hardware processors are configured to execute programmed instructions stored in the one or more first memories, to: identify a first set of nucleotide repeat sequences in the sequenced DNA which are occurring more than a predefined number of times in Pseudomonas aeruginosa; identify a second set of nucleotide repeat sequences in the extracted DNA which are occurring more than a predefined number of times in Staphylococcus aureus; identify a first set of neighborhood genes present upstream and downstream of the first set of nucleotide repeat sequences; identify a second set of neighborhood genes present upstream and downstream of the second set of nucleotide repeat sequences; annotate the first and second set of neighborhood genes according to their functional roles in their respective pathogen based on their involvement in pathways in the identified set of neighborhood genes; and test the presence of a secondary structure in the identified first and second set of nucleotide repeat sequences. The administration module prepares and administers an engineered polynucleotide construct on the infected area depending on the presence of the secondary structure to combat the infections due to Pseudomonas aeruginosa and Staphylococcus aureus, wherein the engineered polynucleotide construct is comprising: one or more of the first and the second set of nucleotide repeat sequences with multiple copies at dispersed locations on the candidate pathogen genomes of one or more of the Pseudomonas or Staphylococcus, wherein the first set of nucleotide repeat sequences comprises a Sequence ID 001 or reverse complement of the sequence ID 001, and the second set of nucleotide repeat sequences comprises one or more of a Sequence ID 002, a Sequence ID 003, reverse complement of the Sequence ID 002 or reverse complement of the Sequence ID 003, a first enzyme capable of nicking and cleaving the identified set of nucleotide sequences, and a second enzyme capable of removal of a set of neighborhood genes flanking the set of nucleotide repeat sequences. The efficacy module checks the efficacy of the administered engineered polynucleotide construct to combat the infections due to Pseudomonas aeruginosa and Staphylococcus aureus after a predefined time period; and re-administers the engineered polynucleotide construct if the Pseudomonas aeruginosa and Staphylococcus aureus are still present in the infected area post administering.

In another aspect, a method for combating infections due to Pseudomonas aeruginosa and Staphylococcus aureus is provided. the method comprising. Initially, a sample is obtained from an infected area. The DNA/RNA is isolated and extracted from the obtained sample using one of a laboratory method. Later, the isolated DNA/RNA is sequenced using a sequencer. In the next step, a first set of nucleotide repeat sequences is identified in the sequenced DNA which are occurring more than a predefined number of times in Pseudomonas aeruginosa. Similarly, a second set of nucleotide repeat sequences is also identified in the extracted DNA which are occurring more than a predefined number of times in Staphylococcus aureus. Further, a first set of neighborhood genes present upstream and downstream of the first set of nucleotide repeat sequences is identified. Similarly, a second set of neighborhood genes present upstream and downstream of the second set of nucleotide repeat sequences is identified. In the next step, the first and second set of neighborhood genes is annotated according to their functional roles in their respective pathogen based on their involvement in pathways in the identified set of neighborhood genes. Later, the presence of a secondary structure is tested in the identified first and second set of nucleotide repeat sequences. Further, an engineered polynucleotide construct prepared and administered on the infected area depending on the presence of the secondary structure to combat the infections due to Pseudomonas aeruginosa and Staphylococcus aureus, wherein the engineered polynucleotide construct is comprising: one or more of the first and the second set of nucleotide repeat sequences with multiple copies at dispersed locations on the candidate pathogen genomes of one or more of the Pseudomonas or Staphylococcus, wherein the first set of nucleotide repeat sequences comprises a Sequence ID 001 or reverse complement of the sequence ID 001, and the second set of nucleotide repeat sequences comprises one or more of a Sequence ID 002, a Sequence ID 003, reverse complement of the Sequence ID 002 or reverse complement of the Sequence ID 003, a first enzyme capable of nicking and cleaving the identified set of nucleotide sequences, and a second enzyme capable of removal of a set of neighborhood genes flanking the set of nucleotide repeat sequences. In the next step, the efficacy of the administered engineered polynucleotide construct is checked to combat the infections due to Pseudomonas aeruginosa and Staphylococcus aureus after a predefined time period. And finally, the engineered polynucleotide construct is re-administered if Pseudomonas aeruginosa and Staphylococcus aureus are still present in the infected area post administering.

The target sites or nucleotide repeat sequences in this disclosure refer to nucleotide sequences which repeat a minimum number of ten times within the genome of the candidate pathogen/pathogens which are identified in an infected site from which the sample is collected. These nucleotide repeat sequences can be targeted in order to debilitate the pathogen. The mentioned nucleotide repeat sequence/sequences is selected if it occurs more than 10 times in all the strains of the candidate specie or genus to which the candidate pathogen/pathogens identified in an infected site belong. The nucleotide repeat sequence is selected such that it does not occur more than twice in genomes of strains belonging to any other genus than that of the candidate pathogen and does not occur more than twice within the genome of the host.

In yet another aspect, one or more non-transitory machine readable information storage mediums comprising one or more instructions which when executed by one or more hardware processors cause combating infections due to Pseudomonas aeruginosa and Staphylococcus aureus is provided. the method comprising. Initially, a sample is obtained from an infected area. The DNA/RNA is isolated and extracted from the obtained sample using one of a laboratory method. Later, the isolated DNA/RNA is sequenced using a sequencer. In the next step, a first set of nucleotide repeat sequences is identified in the sequenced DNA which are occurring more than a predefined number of times in Pseudomonas aeruginosa. Similarly, a second set of nucleotide repeat sequences is also identified in the extracted DNA which are occurring more than a predefined number of times in Staphylococcus aureus. Further, a first set of neighborhood genes present upstream and downstream of the first set of nucleotide repeat sequences is identified. Similarly, a second set of neighborhood genes present upstream and downstream of the second set of nucleotide repeat sequences is identified. In the next step, the first and second set of neighborhood genes is annotated according to their functional roles in their respective pathogen based on their involvement in pathways in the identified set of neighborhood genes. Later, the presence of a secondary structure is tested in the identified first and second set of nucleotide repeat sequences. Further, an engineered polynucleotide construct prepared and administered on the infected area depending on the presence of the secondary structure to combat the infections due to Pseudomonas aeruginosa and Staphylococcus aureus, wherein the engineered polynucleotide construct is comprising: one or more of the first and the second set of nucleotide repeat sequences with multiple copies at dispersed locations on the candidate pathogen genomes of one or more of the Pseudomonas or Staphylococcus, wherein the first set of nucleotide repeat sequences comprises a Sequence ID 001 or reverse complement of the sequence ID 001, and the second set of nucleotide repeat sequences comprises one or more of a Sequence ID 002, a Sequence ID 003, reverse complement of the Sequence ID 002 or reverse complement of the Sequence ID 003, a first enzyme capable of nicking and cleaving the identified set of nucleotide sequences, and a second enzyme capable of removal of a set of neighborhood genes flanking the set of nucleotide repeat sequences. In the next step, the efficacy of the administered engineered polynucleotide construct is checked to combat the infections due to Pseudomonas aeruginosa and Staphylococcus aureus after a predefined time period. And finally, the engineered polynucleotide construct is re-administered if Pseudomonas aeruginosa and Staphylococcus aureus are still present in the infected area post administering.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles:

FIG. 1 illustrates a block diagram of a system for combating infections due to Pseudomonas aeruginosa and Staphylococcus aureus according to an embodiment of the present disclosure.

FIG. 2A and 2B show nucleotide repeat sequences along with neighborhood genes in the Pseudomonas aeruginosa genome and Staphylococcus aureus genome according to an embodiment of the disclosure.

FIG. 3 shows components of a engineered polynucleotide construct containing multiple target nucleotide sequences capable of combating Pseudomonas aeruginosa and Staphylococcus aureus infections according to an embodiment of the disclosure.

FIG. 4 shows targeting of palindromic nucleotide repeat sequences in pathogen genomes according to an embodiment of the disclosure.

FIG. 5 shows enzymatic cleavage in the Pseudomonas aeruginosa and Staphylococcus aureus genomes according to an embodiment of the disclosure.

FIG. 6A-6B is a flowchart illustrating the steps involved in combating infections due to Pseudomonas aeruginosa and Staphylococcus aureus according to an embodiment of the present disclosure.

### DETAILED DESCRIPTION

Exemplary embodiments are described with reference to the accompanying drawings. In the figures, the left-most digit(s) of a reference number identifies the figure in which the reference number first appears. Wherever convenient, the same reference numbers are used throughout the drawings to refer to the same or like parts. While examples and features of disclosed principles are described herein, modifications, adaptations, and other implementations are possible without departing from the scope of the disclosed embodiments. It is intended that the following detailed description be considered as exemplary only, with the true scope being indicated by the following claims.

### GLOSSARY—TERMS USED IN THE EMBODIMENTS

The expression “nucleotide repeat sequence” or “repeated nucleotide sequences” or “repeat sequence” or “the set of nucleotide repeats” or “repeated sequence regions” or “similar sequence stretches” or “target sequence” or “target sites” or “target nucleotide repeat sequence” or “conserved stretch of nucleotide sequences” or “repeat element” in the context of the present disclosure refers to nucleotide sequences or stretches of nucleotide sequences which have been repeated multiple times in a sequence of DNA extracted from a sample obtained from the infected area or within nucleotide sequence obtained for a genomic sequence of a pathogen or genomic sequences of strains belonging to a pathogenic genus or specie.

The term “metagenome” refers to the genetic material derived directly from the infected site and can be considered representative of overall microorganisms present in a sample collected from an environment. The information about metagenome and its taxonomic constitution is obtained by either sequencing the genes considered as markers for different taxa (For example 16S rRNA), amplifying genes of interest using specific primers through methods like but not limited to Polymerase Chain Reaction (PCR). This information can also be obtained by whole genome sequencing of the obtained environmental or metagenomic sample. The sample collected from the environment is referred to from now on as metagenomic sample.

The term “identified repeated nucleotide sequence or ‘identified nucleotide repeat sequence’ is dispersed across distant locations in the pathogen genome” refers to the fact that the nucleotide sequences identified in this method are spread at distant locations across the pathogen genome and is not clustered together at one particular location alone on the genome.

In this disclosure, the terms “distant location” or “distinct location” or “dispersed location” refer to locations of two nucleotide repeat sequences that are separated by more than 10000 base pairs. Nucleotide repeat regions having distance less than 10000 base pairs between their locations have been considered as clustered repeats.

The expression “candidate genus” or “candidate pathogen” refers to the genus, specie or pathogen in which the nucleotide repeat sequence is identified and is used as a target sequence/site.

The term “commensal” refers to microbe/microbes which are considered beneficial to the host or cause no harm to the host.

The term ‘pathogen’ refers to microbe/microbes which cause a disease in host.

The term ‘host’ refers to either a living organism or an environmental site. In an embodiment, ‘host’ may refer to human, animal or plant in which a pathogenic infection may be observed.

The term ‘non-culturable’ refers to microbes that cannot be grown in a laboratory settings because the ideal conditions and media for their growth is not well characterized. Such microbes can be analyzed by culture independent methods discussed in various embodiments of the disclosure.

Majority of the existing methods for combating pathogens focus on silencing specific genes in order to curtail their expression. Targeting single functional aspects of bacteria often is not sufficient as bacteria might mutate the targets and develop resistance to the therapeutic intervention. To overcome the drawbacks of the existing methods, the present system and method deals with identifying and targeting multiple copies of a nucleotide repeat sequence at distant locations on the genome as well as the important functional genes flanking this sequence. Therefore, the method allows to debilitate multiple important functions of the pathogen simultaneously. The important functional genes in this disclosure refer to the genes in pathogens which encode for proteins which are critical for survival, pathogenicity, interaction with the host, adherence to the host or for the virulence of bacteria. Development of resistance in pathogens to the method mentioned in this disclosure is difficult as the pathogen will have to bring about multiple mutations in distant locations. The present disclosure includes targeting multiple virulence and essential proteins of pathogens. The method may also include targeting various other proteins performing important functions (metabolism, host interactions, pathogenicity etc.) in bacteria.

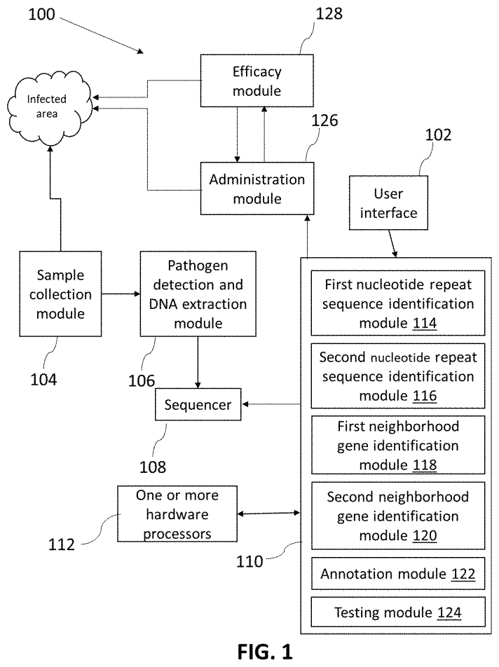

Referring now to the drawings, and more particularly to FIG. 1 through FIG. 6, where similar reference characters denote corresponding features consistently throughout the figures, there are shown preferred embodiments and these embodiments are described in the context of the following exemplary system and/or method.

According to an embodiment of the disclosure, a system 100 for combating infections due to Pseudomonas aeruginosa and Staphylococcus aureus is shown in the block diagram of FIG. 1. The system 100 is configured to provide strategies to combat pathogenic infections caused by multi-drug resistant (MDR) and extensively drug resistant (XDR) strains of Pseudomonas aeruginosa and Staphylococcus aureus. The strategy involves identifying potential target sites in a pathogen, which can be utilized to compromise its multiple virulence or essential functions at the same time. The idea used in this disclosure utilizes the fact that a conserved stretch of nucleotide sequence occurring multiple times on a pathogen genome in genomic neighbourhood of genes encoding virulence factors or in vicinity of genes essential for pathogen survival encoded within the genome of the candidate pathogen can be targeted to disrupt the overall genetic machinery of the pathogen. These nucleotide repeat sequences might also lie in the neighborhood of genes which perform other critical functions in a pathogen. In the present disclosure genomic neighbourhood or vicinity or ‘flanking genes’ refers to regions lying within a predefined number of genes to the selected nucleotide repeat sequence (or its reverse complement) on the nucleotide sequence of the candidate pathogen genome or within a distance of predefined number of bases with respect to the selected nucleotide repeat sequence (or its reverse complement) on the nucleotide sequence of the pathogen genome. The flanking genes are found on each strand on pathogen genomic DNA. In an embodiment the genomic neighbourhood or flanking genes may comprise of 10 genes lying on either side of nucleotide repeat sequence or its reverse complement in terms of its location on the pathogen genome. The important functional genes in this disclosure refer to the genes in pathogens which encode for proteins which are critical for survival, pathogenicity, interaction with the host, adherence to the host or for the virulence of pathogen. The reverse complement of target sequence is obtained by interchanging letters A and T and interchanging letters C and G between target and complement sequence.

A conserved stretch of sequence refers to a nucleotide repeat sequence which occurs within all pathogenic genomes belonging to a candidate genus. Another important factor would be occurrence of these sequences only in the genomic sequences of the pathogenic strains of candidate pathogen and minimum cross reactivity with the commensals (belonging to same candidate genus or other genera) as well as the host. Cross reactivity, in this disclosure, refers to the occurrence of these conserved stretches of nucleotide sequences more than twice in genomes of strains belonging to genera/specie other than the candidate genus/specie or more than twice within commensal bacteria belonging to the candidate genus for which this sequence is being utilized as a target. The nucleotide repeat sequence should not occur more than twice in the host genome also. Further, the identified potential target sites in pathogen are not specific to a single strain of the pathogen. In most cases, metagenomic samples contain bacteria whose strain level information cannot be obtained. Thus, the method can be utilized to target all pathogens strains in the given candidate genus/species of the bacteria and is not hindered by the absence of strain level information.

The present disclosure has been specifically explained on the sequenced genomes of Pseudomonas aeruginosa and Staphylococcus aureus. Both these pathogens are multi drug resistant and responsible for a large part of nosocomial infections across all geographies.

According to an embodiment of the disclosure, the system 100 consists of a user interface 102, a sample collection module 104, a pathogen detection and DNA extraction module 106, a sequencer 108, a memory 110 and one or more hardware processors 112 (referred to as processor 112) as shown in FIG. 1. The processor 112 is in communication with the memory 110. The memory 110 further includes a plurality of modules for performing various functions. The memory 110 may include a first nucleotide repeat sequence identification module 114, a second nucleotide repeat sequence identification module 116, a first neighborhood gene identification module 118, a second neighborhood gene identification module 120, an annotation module 122 and a testing module 124. The system 100 further comprises an administration module 126 and an efficacy module 128 as shown in the block diagram of FIG. 1.

According to an embodiment of the disclosure, the sample is collected from the infected area using the sample collection module 104. In this module, the method utilized for extracting samples from the infected sites depends largely on the site of infection. In an embodiment, in cases of topical infection in a living organism (for example, skin infections caused by Staphylococcus epidermidis and Staphylococcus aureus etc.), the sample is collected from the infected sites such as skin, mucosal lining of tissues such as eyes, mouth and vagina. In another example, the samples may also be obtained from infected area comprising one or more of fecal matter, blood, urine, tissue biopsy, hospital surfaces or environmental samples. Various techniques are used as per the guidance of the physician such as a sterile swab (for example, cotton swabs) for sample collection from the mucosal lining and saliva, a sterile syringe for sample collection from the pus and aspirations of fluids. A skin scrape can also be performed for sample collection from the infected sites on the skin. Also tissue biopsy can be performed in order to obtain the samples.

In an embodiment, in case of blood borne pathogens such as Staphylococcus aureus and Pseudomonas aeruginosa, the sample can be extracted through collection of blood components. Acute serum collected from the patients (containing high concentration of infectious bacteria) can be used. Additionally, the whole blood sample can be submitted for bacterial culturing or the whole blood plasma can be utilized for further procedure.

In an embodiment where the site of infection can also be an environment such as soil, air, water or surfaces (such as infection of Staphylococcus aureus and Pseudomonas aeruginosa in hospital surfaces) etc. Sample collection from a surface can be performed using a sterile swab. Dry swabs may be recommended for wet surfaces and wet swabs are recommended for dry surfaces. Swabbing of the test surface maybe performed by rolling the swab lightly back and forth. Water and soil samples may be collected from the environmental site of infection and sent for further procedure. Air samples can also be collected to identify the presence of air borne pathogen. Volumetric air samples for culture analyses can be taken by impacting a known volume of air onto a suitable growth medium. Any other laboratory accepted method of sample extraction/collection from environment as well as living organisms is within the scope of this invention.

DNA/RNA is isolated and then extracted from the sample using laboratory standardized protocol using the pathogen detection and DNA extraction module 106 and sequencing is performed using the sequencer 108. It should be appreciated, that the bacterial cells are isolated from the extracted sample before being presented to pathogen detection and DNA extraction module 106 in cases where the pathogen is known to be culturable. In case of non-culturable pathogen, the collected samples are directly processed to the pathogen detection and DNA extraction module 106, DNA/RNA is isolated and extracted from the sample using laboratory standardized protocols using the pathogen detection and DNA extraction module 106 and sequencing is performed using the sequencer 108. The nucleotide sequences obtained after sequencing of extracted DNA/RNA sequences are then provided to the processor 112 using the user interface 102. The nucleotide sequences can be obtained for 16S rRNA, a nucleotide sequence encoding for any particular gene of interest being amplified, or sequences corresponding to DNA fragments corresponding to whole genome sequencing or shotgun sequencing. In one embodiment, DNA/RNA can be extracted using DNA isolation and isolation kits such as miniprep and other methods standardized in laboratory setups. The extracted DNA is then provided into the sequencer 108 and the sequences so obtained are fed into the processor 112 using the user interface 102. The user interface 102 is operated by a user. The user interface 102 can include a variety of software and hardware interfaces, for example, a web interface, a graphical user interface, and the like and can facilitate multiple communications within a wide variety of networks N/W and protocol types, including wired networks, for example, LAN, cable, etc., and wireless networks, such as WLAN, cellular, or satellite.

The pathogen detection and DNA extraction module 106 is also configured to utilize experimental techniques to detect pathogens present in an infected site. The use of any laboratory acceptable methods of detecting presence of pathogens present at the infected site is within scope of the disclosure. In one embodiment, presence of viable living cells can be detected by utilizing presence of bacterial mRNA which has a short half-life and will not exist once the cells are dead. This mRNA based method may involve identifying antigen/protein specific for the pathogen which can be utilized as a marker for that pathogen and produced by the pathogen in abundance and the corresponding gene on the pathogen genome can be obtained (For example, Staphylococcal enterotoxin A, leukocidin and Hemolytic toxin in Staphylococcus aureus, Phenazine biosynthesis in Pseudomonas aeruginosa etc). The mRNA corresponding to expression of these genes can be detected using techniques like but not limited to polymerase chain reaction (RT-PCR) assays or reverse transcriptase strand displacement amplification (RT-SDA) assays. In another embodiment, expression of proteins identified as specific to these pathogens can be detected using various laboratory accepted methods for protein purification and detection (For example, toxins in Staphylococcus aureus and Siderophores and phenazine production proteins in Pseudomonas etc.). Chromogenic enzyme assays for a pathogen are also within scope of the invention. Specific metabolites or compounds produced by a pathogen can also be detected (using different laboratory acceptable methods like Mass spectrometry, HPLC-MS, spectrometry-based methods etc.) to ascertain pathogen presence (e.g. Phenazine production in Pseudomonas aeruginosa). In other embodiments, methods like nucleic acid amplification tests (NAAT), real time PCR, immunoassays for the identified antigens as well as specific staining and microscopy techniques and flow cytometry methods of detecting pathogens are also within scope of this invention. PCR or Restriction Fragment Length Polymorphism (RFLP) based detection of 16S rRNA in order to identify pathogens can also be utilized. In one more embodiment, staining methods can also be utilized to identify a pathogen and establish viability of a pathogen cell (e.g. propidium iodide can be used for identifying dead cells). Cell toxicity assays can also be utilized for toxins based detection of pathogens. Further in case of sporulating bacteria, spore detection assays can also be utilized. In case of culturable bacteria, the viability of pathogens can even be established by culturing methods using selective media followed by methods to detect specific pathogens discussed above. In case of an infection in living beings observation of phenotypic effects like alleviation of infection symptoms is also within scope of this disclosure. The symptoms may vary with type of infection and may be observed by registered medical practitioner or healthcare professional. Any other method of detecting pathogens are also within scope of this disclosure.

According to an embodiment of the disclosure, the pathogen detection and DNA extraction module 106 is configured to applying one or more techniques for identification or detection of microbes in a collected sample comprising a sequencing technique, a flow cytometry based methodology, a microscopic examination of the microbes in collected sample, microbial culture of pathogens in vitro, immunoassays, cell toxicity assay, enzymatic, colorimetric or fluorescence assays, assays involving spectroscopic/spectrometric/chromatographic identification and screening of signals from complex microbial populations, The pathogen or microbial characterization data may comprise one or more of sequenced microbial DNA data, a Microscopic imaging data, a Flow cytometry cellular measurement data, a colony count and cellular phenotypic data of microbes grown in in-vitro cultures, immunological data, proteomic/metabolomics data, and a signal intensity data. The sequenced microbial data obtained from sequencer 108 comprises sequences obtained from next generation sequencing platforms comprising one or more of marker genes including 16S rRNA, Whole Genome Shotgun (WGS) sequences, a fragment library based sequences, a mate-pair library or a paired-end library based sequencing technique, or a combination thereof. The sequencing data may also comprise of complete genome sequences of the pathogens obtained within a collected sample. In one embodiment, the taxonomic groups or pathogens within a sample collected can be obtained by amplification of marker genes like 16S rRNA within bacteria. In another embodiment, the taxonomic groups or pathogens within a sample can be obtained by the binning of whole genome sequencing reads into various taxonomic groups using different methods including sequence similarities as well as several methods using supervised and unsupervised classifiers for taxonomic binning of metagenomics sequences.

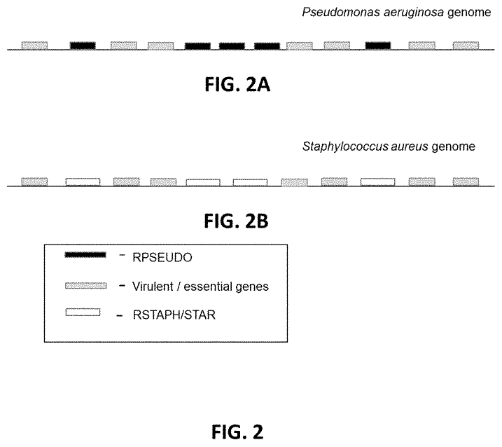

According to an embodiment of the disclosure, the memory 110 comprises the first nucleotide repeat sequence identification module 114 and the second nucleotide repeat sequence identification module 116. The first nucleotide repeat sequence identification module 114 is configured to identify a first set of nucleotide repeat sequences in the extracted DNA which occur more than a predefined number of times (refers to the number of occurrences of nucleotide repeat sequence on a genome in a dispersed manner and this number might vary with system and pathogen under consideration) in the genomic sequences of different strains of Pseudomonas aeruginosa and are dispersed at distant locations on the genome. The predefined number refers to the number of occurrences of nucleotide repeat sequence on genomic sequences of all pathogenic strains of candidate pathogens in a dispersed manner and this number might vary with system and pathogen under consideration. A minimum of 10 occurrences is required for a nucleotide repeat sequence to be considered. In an example, RPSEUDO is identified as shown in schematic representation in FIG. 2A. The second nucleotide repeat sequence identification module 116 is configured to identify a second set of nucleotide repeat sequences in the extracted DNA which occur more than a predefined number of times in the genomic sequences of pathogenic strains of Staphylococcus aureus and are dispersed at distant locations on the genome. In an example, STAR element or RSTAPH is identified as shown in schematic representation of FIG. 2B. Further, it is important to ensure that the identified first and second nucleotide repeat sequence region is specific to a particular candidate pathogenic genus only (Pseudomonas and Staphylococcus here) and, on nucleotide sequence based alignment, shows no more than two cross matches with commensals of the other genera or commensals within same genus (Staphylococcus and Pseudomonas here). Cross match refers to the occurrence of identified nucleotide repeat sequence region more than two times in a genus which is different from the candidate genus in which the nucleotide repeat sequence has been identified as is to be used as a target site.

In addition to that, the identified first set and the second set of nucleotide repeat sequences are not specific to a single strain of the pathogen. For example, RPSEUDO is present in multiple strains of Pseudomonas aeruginosa and RSTAPH is present in multiple pathogenic strains of Staphylococcus aureus. In most cases, metagenomic samples contain bacteria whose strain level information cannot be obtained. Thus, the method can be utilized to target all pathogens in the given species of the bacteria and is not hindered by the absence of strain level information and making it more robust.

Following method can be used for the identification of the nucleotide repeat sequence region.

Conserved nucleotide repeat elements were identified on Pseudomonas and Staphylococcus aureus genomes by taking nucleotide sequence stretches of predefined length Rn (30-35 in this embodiment for Pseudomonas aeruginosa and 20-25 for Staphylococcus aureus), picked from the genome sequence of candidate pathogen or different strains of candidate pathogen (Pseudomonas aeruginosa and Staphylococcus aureus in this disclosure), keeping the difference in the start position of consecutive picked nucleotide stretches Rnᵢ₊₁ and Rnᵢ as 5 nucleotides. Predefined length Rn refers to the length of a stretch of nucleotide sequence (picked from the complete nucleotide sequence of a bacterial genome) used as a seed input for local sequence alignment tools. This predefined length may differ depending on the pathogen

In the present embodiment for Pseudomonas aeruginosa, stretches of sequences were aligned within the genome itself by local alignment (as implemented in PILER software) to find the location of these elements in all sequenced Pseudomonas genomes. Sequence based search utilizing any other sequence alignment (e.g. Burrows Wheeler alignment) or repeat finding tools are within scope of this invention. Sequence based search utilizing BLAST can also be utilized for this purpose. In the next step, a reference genome based nucleotide sequence alignment tool is applied in order to align the picked nucleotide sequence stretch with nucleotide sequences corresponding to genomes of all pathogenic strains belonging to the candidate pathogen, genus or specie. A relaxation of two mismatches was allowed to prevent false positives which could lead to over-prediction of possible targets. Similar methods were utilized for identification of nucleotide repeat sequences in Staphylococcus genomes. Nucleotide repeat sequences Rn occurring more than 30 times at distant locations on the genome were considered. This number of occurrences may vary depending on the system requirements but a minimum of 10 occurrences is required for a nucleotide repeat sequence to be considered as a target sequence. The nucleotide repeat sequence RPSEUDO was obtained in Pseudomonas aeruginosa while two sets of nucleotide sequences RSTAPH and STAR were obtained in Staphylococcus aureus. The dispersed nucleotide sequences at distant locations on the genome refers to stretches of nucleotide sequences which occur across the genome with a distance of predefined number of base pairs between them. In one embodiment used in this disclosure the predefined number refers to a separation of >10000 base pairs between two nucleotide repeat sequences. If the number of times Rₙ matches on the genomic sequences of strains of candidate pathogen genome/genomes is greater than the predefined threshold with a minimum value of 10, the nucleotide sequence stretch is termed as target nucleotide repeat sequence. The nucleotide repeat sequences which are conserved across all genome sequences corresponding to strains of a candidate pathogen or genus would indicate the said conserved sites. Any other method of identification of conserved sites is also within the scope of this disclosure.

According to an embodiment of the disclosure, the memory 110 further includes the first neighborhood gene identification module 118 and a second neighborhood gene identification module 120. The first neighborhood gene identification module 118 is configured to identify a first set of neighborhood genes present upstream and downstream of the first set of nucleotide repeat sequences (on the nucleotide sequence on the genome of the candidate pathogen) corresponding to Pseudomonas aeruginosa. The second neighborhood gene identification module 120 is configured to identify a second set of neighborhood genes present upstream and downstream (on the nucleotide sequence on the genome of the candidate pathogen of the second set of nucleotide repeat sequences corresponding to Staphylococcus aureus. On each Pseudomonas genome where nucleotide repeat elements or its reverse complement occur, 10 flanking genes both upstream and downstream were found on each strand (+and −) of DNA. Similarly, 10 flanking genes upstream and downstream of the nucleotide repeat elements or its reverse complement were also identified on each Staphylococcus genome. The number of flanking genes considered may vary with the system.

According to an embodiment of the disclosure, the system 100 further includes the annotation module 122. The annotation module 122 categorizes or annotates the first set and the second set of neighborhood genes based on their functional roles in the pathogen. Functional annotation of these genes was performed using HMM search with PFAM as the database. In other embodiments, databases like CDD, SMART etc. can be utilized. The use of any other methods such as PSSM, BLAST etc. is well within the scope of the disclosure.

These dispersed nucleotide repeat sequences RPSEUDO, RSTAPH and STAR at distant locations on the genome can be used as targets which can be further extended to target multiple flanking genes (which includes virulence and survival genes) simultaneously at distant multiple locations and carry out changes like but not limited to gene silencing, gene recombination, gene substitution with a new function etc.

Functional categorization of these genes on the basis of pathways they are involved in was carried out using literature mining. The broad categories have been discussed in Table 1 and Table II.

TABLE 1
Summary of proteins in vicinity conserved sequence
RPSEUDO in Pseudomonas aeruginosa
Essential Proteins

| | | |
|---|---|---|
| Metabolism | Fatty acyl CoA | Involved in |
| | dehydrogenases Fad | metabolizing variety |
| | proteins | of fatty acids |
| | Fructose gene cluster | Utilization of fructose |
| | Glucose dehydrogenase | Glucose metabolism |
| | Glycerol gene cluster | Glycerol metabolism |
| | NadE protein | Nicotinamide |
| | | biosynthesis |
| Nucleotide | Pur gene cluster | Purine biosynthesis |
| biosynthesis | Cystosine biosynthesis | Pyrimidine |
| | | biosynthesis |
| Transcription | Transcriptional | Multiple gene clusters |
| regulation | regulators | |
| Cell wall | D-Alanine ligase Muramic | Peptidoglycan layer |
| biosynthesis | acid biosynthesis | |

Virulence/Pathogenic proteins

| | | |
|---|---|---|
| Biofilms | Las and Rhl genes | Homoserine lactones |
| | Phenazine gene clusters | Phenazine molecules |
| | Phh gene cluster | Phenylalanine |
| | | metabolism |
| | Pyoverdine gene cluster | Siderophore biofilms |
| | GGDEF | c-di-GMP biosynthesis |
| | Chemotactic proteins | chemotaxis |
| | Type III secretion | Biofilm 2nd stage |
| | Two component Syetems | Signalling |
| Antibiotic | Efflux pumps | Multidrug resistance |
| resistance | Vanillate porins | Vanillate efflux |
| Stress response | RNAases and helicases etc. | Repair machinery |
| | Clp protease | Stress response |

TABLE 2
Summary of proteins in vicinity of repeat
elements in Staphylococcus genomes

| Category | Annotated Genes | Function |
|---|---|---|
| Toxins | Staphylococcal toxin | Causes toxic shock |
| | | syndrome leading to |
| | | vomiting and diarrhea |
| | Staphylococcal | Lyse the host red blood |
| | haemolysin protein | cells |
| | Leukocidin | Lyse the host white |
| | | blood cells |
| | Exfoliative | Serine proteases that |
| | toxin A/B | cause blistering on the |
| | | skin. |
| Biofilm | CHAP proteins | Involved in |
| Formation | | peptidoglycan hydrolysis |
| | | during biofilm formation |
| | Ica Cluster | Secretes inter-cellular |
| | (A/B/C/D) | adhesion proteins |
| | Que Cluster | Queuosine biosynthesis |
| | (C/D/E/F) | |
| Antibiotic | Vra R/S/SR | Vancomycin resistance |
| Resistance | | |
| Host Immune | Urease Cluster | Molecular mimicry, |
| Evasion | (Urease α/β/γ) | immunogenic response |
| | | in host, alternate |
| | | nitrogen metabolism, |
| | | evasion from |
| | | macrophages |
| | SCIN | Evasion from host |
| | (Staphylococcal | complement system |
| | complement | |
| | inhibitor protein) | |
| DNA Repair | Uvr Cluster | Excision repair system |
| machinery | (A/B/C/D) | |
| | DNA Topoisomerase | Unwinding or rewinding |
| | | DNA supercoils during |
| | | repair. |
| Competence | ComFA | Uptake of extracellular |
| Protein | | DNA |
| Essential | Muramyl ligase | Involved in |
| Proteins | | peptidoglycan layer |
| | | formation |
| | Sugar transporters | Uptake of glucose and |
| | | other carbohydrate |
| | | sources by the bacteria |
| | Mannose-6- | Involved in glycolysis |
| | phosphate | |
| | isomerase | |

According to an embodiment of the disclosure, the memory 110 further includes the testing module 124. The testing module 124 is configured to check the presence of secondary structure formation in the identified first and second set of nucleotide repeat sequences. There could be the presence of the secondary structures such as hairpin loop formation.

Depending on the presence of the secondary structure, the administration module 126 is configured to prepare and administer an engineered polynucleotide construct on the infected area depending on the presence of the secondary structure to combat the infections due to Pseudomonas aeruginosa and Staphylococcus aureus, wherein the engineered polynucleotide construct is comprising: one or more of the first and the second set of nucleotide repeat sequences with multiple copies at dispersed locations on the candidate pathogen genomes of one or more of the Pseudomonas or Staphylococcus, wherein the first set of nucleotide repeat sequences comprises a Sequence ID 001 or reverse complement of the sequence ID 001, and the second set of nucleotide repeat sequences comprises one or more of a Sequence ID 002, a Sequence ID 003, reverse complement of the Sequence ID 002 or reverse complement of the Sequence ID 003, a first enzyme capable of nicking and cleaving the identified set of nucleotide sequences, and a second enzyme capable of removal of a set of neighborhood genes flanking the set of nucleotide repeat sequences The engineered polynucleotide construct works in such a way that it targets multiple regions in the genome simultaneously.

In an embodiment the engineered polynucleotide construct may comprise of an engineered circular DNA comprising of an origin of replication. Further the engineered polynucleotide construct may comprise of regulatory elements including a promoter sequence, ribosomal binding site, start codon, a cassette comprising of first and second enzyme flanking the nucleotide repeat sequence or the reverse complement of the nucleotide repeat sequence RPSEUDO/RSTAPH cloned into the system, stop codons and transcription terminator. The promoter sequence may depend on the pathogen being targeted as well as the regulation required to express the components of the engineered polynucleotide construct at a specific targeted site (for example, within a living being or an infected area). The engineered polynucleotide construct may also be equipped to create a poly A tail in mRNA to stabilize the sequence. The poly A tail refers to a stretch of polynucleotide Adenine nucleotides at the 3′ end of mRNA. In one embodiment, the first and second enzyme can be nickase and exonuclease cloned in any order. The target RPSEUDO/RSTAPH within the pathogen genome can be recognized and bound by the reverse complement sequence and the complex thus formed can be nicked by the nickase enzyme. The exonuclease can then cut the duplex formed as well as flanking genes once it recognizes a nick. In another embodiment, the enzymes can be cas9 sequences (may be obtained from Streptococcus pyogenes) flanking the RPSEUDO/RSTAPH sequence or flanking the reverse complement of RPSEUDO/RSTAPH which can both act as sgRNA (single guide RNA) for the obtained CRISPR-Cas (Clustered Regularly Interspaced Short Palindromic Repeats) system. The reverse complement of target nucleotide repeat sequence is obtained by interchanging letters A and T and interchanging letters C and G between target and complement sequences. The reverse complement refers to the sequence corresponding to the identified nucleotide repeat sequence in the opposite strand of DNA. The RPSEUDO/RSTAPH or its reverse complement is recognized by the reverse complement sequence or the target sequence on the engineered polynucleotide construct and the complex formed by the binding of RPSEUDO/RSTAPH sequence to its reverse complement. The cas9 may then act as an endonuclease and cut the nick and flanking sequences. The nucleotide repeat sequence can be targeted by delivering the engineered polynucleotide construct using a bacterial, plasmid or a viral vector to the target bacterial cell. In one embodiment the composition may comprise of: the first element comprising a polynucleotide sequence of CRISPR-Cas system wherein the polynucleotide sequence may comprise a nucleotide repeat sequence (identified repeat or its reverse complement) called a guide sequence capable of hybridizing to target sequence (repeat sequence on pathogen), a tracr sequence and a tracr mate sequence. The second element may comprise of CRISPR enzyme coding sequences like CAS enzymes. It should be noted that in all these embodiments RSTAPH/RPSEUDO sequences can be cloned within same polynucleotide sequence along with a bacterial or viral vector and the other features mentioned above to target more than one pathogen using the same compact engineered polynucleotide construct. Any other construct cassette that may bring about the recognition of the RSTAPH/RPSEUDO sequences in bacterial genomes and subsequent nicking and cutting of RSTAPH/RPSEUDO sequences and the flanking genes is within the scope of this invention.

In another embodiment, in addition to the above mentioned features, if bacterial conjugation is to be used as a construct delivery method, the engineered polynucleotide construct may comprise of a relaxase, coding sequences for structural proteins (e.g. pili) and those for regulatory proteins for conjugation. It should be noted that in both embodiments multiple RPSEUDO/RSTAPH sequences can be cloned to target more than one pathogen using the same compact engineered polynucleotide construct. Any other engineered polynucleotide construct cassette that may bring about the recognition of the RPSEUDO/RSTAPH and subsequent cutting of RPSEUDO/RSTAPH and the flanking genes is within the scope of this invention. These polynucleotides comprising the nucleotide repeat sequence, the genes encoding enzymes and the other features discussed above can be inserted into laboratory acceptable vectors which allow insertion of external DNA fragments. In one embodiment construct may be carried by vectors like plasmid or phage based cloning vectors. The regulatory elements can be designed according to information available for the pathogen being targeted.

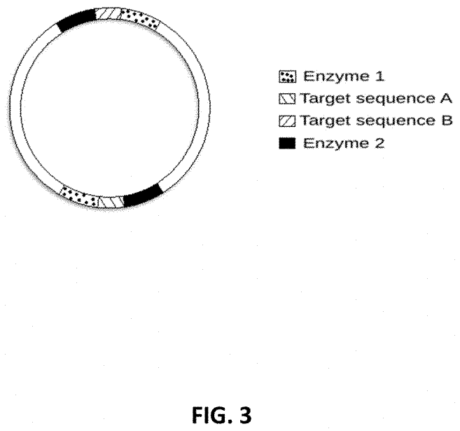

In one embodiment, the engineered polynucleotide construct may contain an enzyme 1, enzyme 2, identified first target sequence (RSTAPH/RPSEUDO) and the identified second target sequence (RSTAPH/RPSEUDO) as shown in FIG. 3. One of the enzyme 1 or enzyme 2 can be the nicking enzyme while the other will constitute nucleotide cleaving enzymes such as nuclease, exo-nuclease etc. Other enzymes with similar activities are also within scope of the invention. The engineered polynucleotide construct with RPSEUDO as well as RSTAPH as target sequences can be used to target both pathogens simultaneously.

Depending on the result of testing module 124, there could be two cases as follows:

Case I: If the identified nucleotide repeat sequences are found to be palindromic the following three strategies may be used.

Strategy I includes handling hairpin loops which hinders DNA transcription by stalling the RNA polymerase enzyme thereby down-regulating the flanking gene expression. In an embodiment, the strategy would involve use of the identified nucleotide repeat sequences as target and inserting a strong palindromic sequence to ensure the down-regulation of transcription of flanking genes

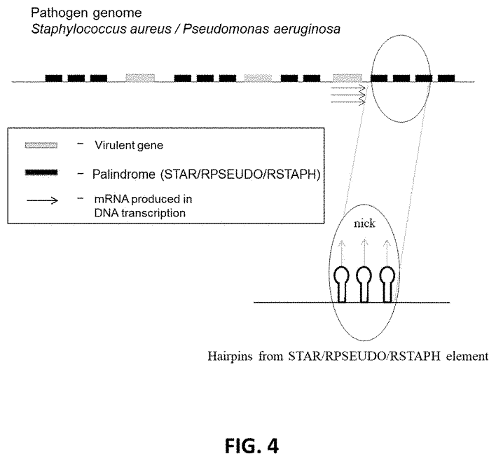

Strategy II involves handling hairpin loops formed in the mRNA which could be involved in prevention of the early decay of mRNA thereby promoting the expression of important bacterial genes. In an embodiment, the strategy may include use of the identified nucleotide repeat sequences as target to nick the pathogen genome at multiple locations and cleave the flanking genes. In an example, a schematic representation of the Pseudomonas/Staphylococcus genome showing nick of Hairpins from STAR element is shown in FIG. 4.

Strategy III is utilized if the identified nucleotide repeat sequences is found to be a transcription terminator and is followed by a polyA tail. In an embodiment, the identified nucleotide repeat sequence is used as target and a strong palindromic sequence is inserted to ensure that the transcriptional termination of the flanking genes occur and these genes are down-regulated in the pathogen.

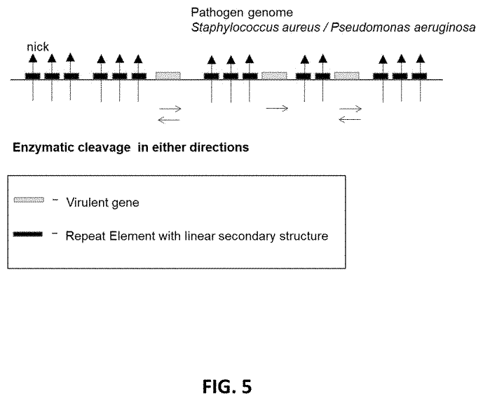

Case II: If the identified nucleotide repeat sequences are not found to be palindromic, the identified repeat sequences are used as target to nick the pathogen genome at multiple locations and cleave the flanking genes. A schematic representation of Pseudomonas/Staphylococcus genome showing enzymatic cleavage in either directions is shown in FIG. 5.

In the present embodiment, the RPSEUDO, STAR element and RSTAPH sequences, are palindromic and may form a hairpin loop structure indicating their role in regulation of transcription. These loops may either form at DNA level or at the ends of their mRNA during DNA transcription. This hairpin loop in the mRNA could be involved in prevention of the early decay of mRNA, resulting in higher protein formation of the virulence genes which are in the vicinity of these palindromic elements. Reduction in pathogenicity can be achieved by decreasing the stability of mRNA corresponding to these virulent genes which can be attained by removing the hairpin loops. If hairpin loop formation takes place at DNA level it might regulate DNA supercoiling and concatenation. The hairpin loop is not followed by a polyA tail indicating it might not be working as transcription terminator.

The administration module 126 can use any pharmaceutically acceptable method of carrying the engineered polynucleotide construct to target the conserved sequences in a pathogen genome. In different embodiments the utility can be, but not limited to oral medicine, topical creams, nasal administration, aerosol sprays, injectable cocktail etc.

In an embodiment, the engineered polynucleotide construct can be administered to the infected site (either living beings or environmental site) through targeted construct delivery methods such as the use of targeted liposomes (wherein, the liposome is tagged on the external surface with molecules that may be specific and functionally important to the candidate genus and the tagged liposome can be used to transfer the engineered polynucleotide construct into the pathogen), targeted nanoparticles wherein, a targeting molecule that is specific to the candidate genus can be attached to the nanoparticle (like but not limited to Ag or Au nanoparticle) along with the engineered polynucleotide construct, thereby allowing the tagged nanoparticle to release the engineered polynucleotide construct into the pathogen, phage based delivery method (wherein, the engineered polynucleotide construct can be placed within the phage infecting the candidate genus thereby transferring the engineered polynucleotide construct into pathogen) and bacterial conjugation (wherein, the engineered polynucleotide construct can be placed in other bacteria that can conjugate with the candidate genus and the engineered polynucleotide construct can be transferred to the pathogen through natural conjugation method) etc. In an embodiment, the lipid constitution of the membrane for the targeted liposome can be modified to target specific set of bacteria. In one example, liposomes containing lipids like Dipalmitoyl phosphatidyl Choline (DPPC) and cholesterol can lead to release of the engineered polynucleotide construct within contained the liposome after encountering rhamnolipids which are prevalent in Pseudomonas aeruginosa biofilms. Similarly, cationic liposomes with lipid constitution comprising dioctadecyldimethylammonium bromide (DDAB) may be used to target Staphylococcus biofilms. In another example, Staphylococcus aureus biofilms are targeted by utilizing antigens like Wheat Germ agglutinin as ligands on nanoparticles to specifically penetrate and bind to S. aureus.

In another embodiment, immunoliposomes can be created with specific antibodies towards ligands of specific pathogen (for example, antibodies against concanavalin A for targeting extracellular matrix of biofilms). The lipid bilayer can be made sensitive to the toxins or other virulence factors of the pathogen in order to release the engineered polynucleotide construct only in infected areas where toxins are present.

In another embodiment, the engineered polynucleotide construct can also be administered to the infected site through non-targeted construct delivery methods such as the use of non-targeted nanoparticles (wherein, nanoparticles can form cages that can hold the engineered polynucleotide construct which are then released into the pathogen), non-targeted liposomes (wherein, the liposomes are phospholipid capsules which can be used to hold the engineered polynucleotide construct that can then merge with the pathogen cell membrane to release the engineered polynucleotide construct inside the pathogen) etc. In an embodiment, attenuated bacteria can also be used to deliver nanoparticles into tissue spaces where they can be released to act upon actual site of infection (as shown in creation of NanoBEADS in a study where Salmonella was used to deliver nanoparticles containing a drug to deep tissues). In another example, minicells produced by bacteria can also be used to package the engineered polynucleotide construct and deliver it to specific areas in the infected site. In another embodiment, these delivery methods can be used to target the engineered polynucleotide construct to infected surfaces also. Any other laboratory accepted method of administration of the engineered polynucleotide construct to the infected site is within the scope of this disclosure.

According to an embodiment of the disclosure, the efficacy module 128 is used to assess the efficacy of the treatment methodology described in this disclosure. The efficacy module 128 comprises of any laboratory acceptable methods of detecting presence of pathogens present at the infected site. In one embodiment, presence of viable living cells can be detected by utilizing presence of bacterial mRNA which has a short half-life and will not exist once the cells are dead. This mRNA based method may involve identifying antigen/protein specific for the pathogen which can be utilized as a marker for that pathogen and produced by the pathogen in abundance and the corresponding gene on the pathogen genome can be obtained (For example, A and B toxins in Clostridium, Staphylococcal enterotoxin A, leukocidin and Hemolytic toxin in Staphylococcus aureus, Phenazine gene cluster in Pseudomonas aeruginosa etc.). The mRNA corresponding to expression of these genes can be detected using techniques like but not limited to polymerase chain reaction (RT-PCR) assays or reverse transcriptase strand displacement amplification (RT-SDA) assays. In another embodiment, expression of proteins identified as specific to these pathogens can be detected using various laboratory accepted methods for protein purification and detection (For example, toxins in Staphylococcus aureus and Siderophores and phenazine production proteins in Pseudomonas etc.). Chromogenic enzyme assays for a pathogen are also within scope of the invention. Specific metabolites or compounds produced by a pathogen can also be detected (using different laboratory acceptable methods like Mass spectrometry, HPLC-MS, spectrometry-based methods etc.) to ascertain pathogen presence (e.g. Phenazine production in Pseudomonas aeruginosa). In other embodiments, methods like nucleic acid amplification tests (NAAT), real time PCR, immunoassays for the identified antigens as well as specific staining and microscopy techniques and flow cytometry methods of detecting pathogens are also within scope of this invention. PCR or Restriction Fragment Length Polymorphism (RFLP) based detection of 16S rRNA in order to identify pathogens can also be utilized. In one more embodiment, staining methods can also be utilized to identify a pathogen and establish viability of a pathogen cell (e.g. propidium iodide can be used for identifying dead cells). Cell toxicity assays can also be utilized for toxins based detection of pathogens. Further in case of sporulating bacteria, spore detection assays can also be utilized. In case of culturable bacteria, the viability of pathogens can even be established using culturing methods based on selective media followed by methods to detect specific pathogens discussed above. In case of an infection in living beings observation of phenotypic effects like alleviation of infection symptoms is also within scope of this disclosure. The symptoms may vary with type of infection and may be observed by registered medical practitioner or healthcare professional. Any other method of detecting pathogens are also within scope of this disclosure. In case pathogen presence is detected, the engineered polynucleotide construct can be administered again using administration module 126 and repeated till pathogen is eliminated.

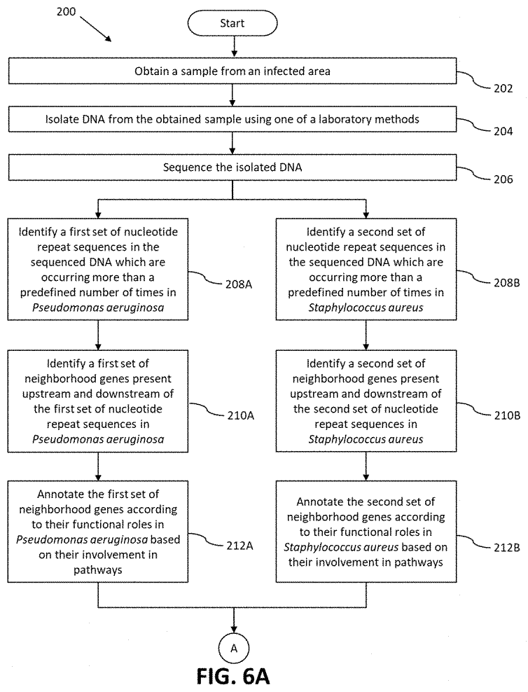

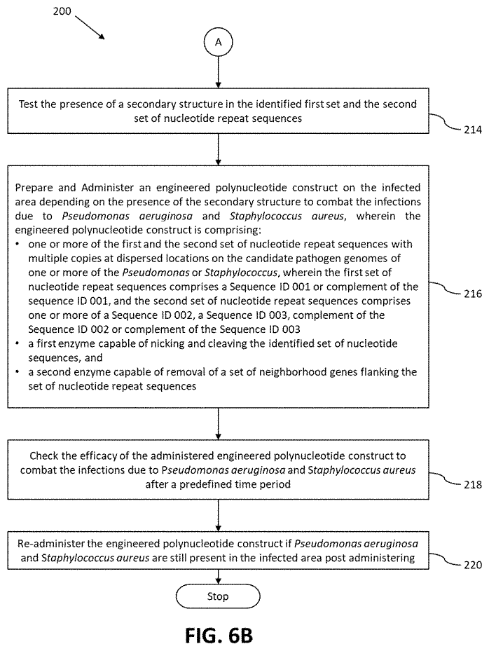

In operation, a flowchart 200 illustrating the steps involved for combating infections due to Pseudomonas aeruginosa and Staphylococcus aureus can be shown in FIG. 6A-6B. Initially at 202, a sample is obtained from an area infected from the pathogen Pseudomonas aeruginosa and Staphylococcus aureus. At step 204, DNA is isolated and extracted from the obtained sample using the pathogen detection and DNA extraction module 106 which is configured for pathogen detection. At step 206, the isolated DNA is sequenced using the sequencer 108. In the next step 208A, the first set of nucleotide repeat sequences in the extracted DNA is identified which occur more than a predefined number of times (refers to the number of occurrences of nucleotide repeat sequence on a genome in a dispersed manner and this number might vary with system and pathogen under consideration where minimum value of predefined number is 10 in the Pseudomonas aeruginosa. In an example, the identified set of nucleotide repeat sequences correspond to RPSEUDO. In addition to that, the identified the first set and the second set of nucleotide sequences are not specific to a single strain of the pathogen. Similarly at next step 208B, the second set of nucleotide repeat sequences in the extracted DNA is identified which occur more than a predefined number of times (refers to the number of occurrences of nucleotide repeat sequence on a genome in a dispersed manner and this number might vary with system and pathogen under consideration, where minimum value of predefined number is 10) in the Staphylococcus aureus. In an example, the identified set of nucleotide repeat sequences correspond to STAR and RSTAPH. In addition to that, the identified the first set and the second set of nucleotide sequences are not specific to a single strain of the pathogen. At step 210A, the first set of neighborhood genes present upstream and downstream of the first set of nucleotide repeat sequences was identified. Similarly at step 210B, the second set of neighborhood genes present upstream and downstream of the second set of nucleotide repeat sequences were also identified.

In step 212A, the first set of neighborhood genes is categorized or annotated according to functional roles of each of neighborhood gene in the Pseudomonas aeruginosa. Similarly, at step 212B the second set of neighborhood genes is categorized or annotated according to functional roles of each of neighborhood gene in the Staphylococcus aureus. At step 214, the presence of the secondary structure is tested in the first and the second set of nucleotide repeat sequences. The first and the second set of nucleotide repeat sequences may be palindromic in nature which may result in the formation of hairpin loops. At step 216, the engineered polynucleotide construct is administered on the infected area depending on the presence of the secondary structure to treat the infection generated due to Pseudomonas aeruginosa and Staphylococcus aureus.

At step 216, an engineered polynucleotide construct is prepared and administered on the infected area depending on the presence of the secondary structure to combat the infections due to Pseudomonas aeruginosa and Staphylococcus aureus, wherein the engineered polynucleotide construct is comprising:
one or one or more of the first and the second set of nucleotide repeat sequences with multiple copies at dispersed locations on the candidate pathogen genomes of one or more of the Pseudomonas or Staphylococcus, wherein the first set of nucleotide repeat sequences comprises a Sequence ID 001 or complement of the sequence ID 001, and the second set of nucleotide repeat sequences comprises one or more of a Sequence ID 002, a Sequence ID 003, complement of the Sequence ID 002 or complement of the Sequence ID 003,
a first enzyme capable of nicking and cleaving the identified set of nucleotide repeat sequences, and
a second enzyme capable of removal of a set of neighborhood genes flanking the set of nucleotide repeat sequences;

The administration of construct aims at targeting the set of identified nucleotide repeats and removal of flanking genes on genomes of pathogen infecting the area. The engineered polynucleotide construct works in such a way that it targets multiple regions in the pathogenic genome simultaneously. At step 218, the efficacy of the administration module is assessed and in case pathogen presence is detected at the site, administration module can be utilized repetitively till Pseudomonas aeruginosa and Staphylococcus aureus is eliminated from the site. And finally at step 220, the engineered polynucleotide construct is re-administered if the Pseudomonas aeruginosa and Staphylococcus aureus are still present after checking using efficacy module 128 in the infected area.

According to an embodiment of the disclosure, the system 100 can also be used in combination with various other known methods to effectively treat the pathogenic infection. In an example, the method 200 can be used as preventive method. The method can be used in combination with various other antibacterial agents. One implementation would be the use of quorum quenchers along with the engineered polynucleotide construct to tackle the biofilm formation in hospital surfaces. In another example, the method may be used as a therapeutic measure. The method may be used in combination with various other antimicrobial methods. One implementation would be to use the method along with antibiotics and vaccines against essential proteins for therapeutic purposes.

Nucleotide repeat elements were identified on sequenced Pseudomonas genomes by taking a nucleotide sequence stretch of predefined length Rn and searching across the genome for similar nucleotide sequence stretches as taught by several alignment software. Nucleotide repeat sequence elements RPSEUDO were identified to be the sequence:

GGCGNATAACNNCN₍₂₋₄₎GNNGTTATNCGCC.

Results of sequence similarity analysis (using BLAST in this embodiment) revealed that this sequence doesn't show any significant nucleotide level sequence similarity in any other bacterial genus or other species of Pseudomonas other than Pseudomonas aeruginosa and showed no significant similarity match with the host human genome nucleotide sequence reducing the possibility of a cross-reactivity. Hence, these elements are ideal candidates for targeting pathogenic Pseudomonas aeruginosa.

A similar approach was used to determine nucleotide repeat sequences in Staphylococcus aureus. Two potential targets were found as discussed below

Firstly, A GC rich repeat sequence of length 15-20 nucleotides was observed. They occur from 30 to 80 times on distinct locations on the genome. Literature evidence points out that these nucleotide repeat regions are previously identified as STAR elements (Staphylococcus aureus repeat elements) and are present in various locations in highly pathogenic Staphylococcus aureus. Further, a modified consensus nucleotide sequence for STAR elements was observed than previously reported. The modified consensus sequence is reported as below:

GTTG(N)₀₋₅(GC)₀₋₆(N)₀₋₅CAAC

where N is any nucleotide.

Secondly, another set of nucleotide repeat regions that are quite different from STAR elements is also identified. This nucleotide sequence stretch, RSTAPH is 53 nucleotides long and occurs from 10 to 15 times on the genome. The consensus nucleotide repeat sequence is reported as below:

GGTGGGACGACGAAATAAATTTTGCGAAAATATCATTTCTGTCC
CACTCCCAA

On further analysis as discussed below, it was observed that these conserved stretches are found in the vicinity of highly virulent and, certain essential genes of Staphylococcus aureus. Results of sequence similarity analysis showed that these element is highly specific to pathogenic Staphylococcus species and are absent in commensals and non-pathogenic or mildly-pathogenic species such as Staphylococcus carnosus and Staphylococcus saprophyticus respectively. Further, these elements don't show any significant sequence similarity in any other bacterial genus and on the host genome, reducing the possibility of a cross reactivity. Hence, these elements are ideal candidates for targeting pathogenic Staphylococcus species.

Another observation made was that a number of small proteins of length 20-100 amino acids, flanked by highly virulent or essential genes of Staphylococcus aureus, were indeed STAR elements. The high GC rich content and the presence of a start and stop codon has resulted in false prediction of these ORFs.

Following is the number of occurrences and locations of STAR repeats in the strains from Staphylococcus aureus is as follows. Due the large number of available strains, only few are provided below:

GCA_000237125.1_Staphylococcus_aureus_subsp._aureus_M013_strain=M013
Number of occurrences: 65

(183925, 183941), (183984, 184000), (294154, 294167), (369298,369311), (421520, 421536), (617059, 617072), (769658, 769673), (769716, 769730), (779101, 779114), (779157, 779170), (779216, 779229), (810688, 810702), (810745, 810758), (816007, 816020), (825058, 825071), (825114, 825129), (825172, 825186), (861492, 861505), (881618, 881633), (926501, 926515), (926507, 926521), (926558, 926571), (1145422, 1145436), (1145474, 1145487), (1149440, 1149453), (1149496, 1149509), (1149552, 1149565), (1149613, 1149628), (1149666, 1149681), (1149672, 1149687), (1286630, 1286643), (1286686, 1286701), (1286692, 1286707), (1664793, 1664808), (1665038, 1665053), (1680369, 1680384), (1680375, 1680390), (1680427, 1680440), (1700149, 1700165), (1700207, 1700220), (1700262, 1700275), (1786078, 1786093), (1788351, 1788367), (1788408, 1788422), (1978692, 1978705), (1978749, 1978762), (1989501, 1989517), (1989558, 1989571), (1989614, 1989627), (2028428, 2028444), (2034183, 2034196), (2038516, 2038531), (2045699, 2045712), (2124373, 2124389), (2124379, 2124395), (2124437, 2124451), (2124652, 2124668), (2124658, 2124674), (2203611, 2203628), (2286012, 2286029), (2286071, 2286084), (2320383, 2320396), (2745307, 2745320), (2745363, 2745376), (2782430, 2782443)

GCA_000737615.1_Staphylococcus_aureus_subsp._aureus_SA268_strain=SA268
Number of occurrences: 62

(174608, 174624), (174667, 174683), (284836, 284849), (359984, 359997), (412206, 412222), (606867, 606880), (759539, 759554), (759597, 759611), (768982, 768995), (769038, 769051), (769097, 769110), (800570, 800584), (800627, 800640), (805889, 805902), (814940, 814953), (814996, 815011), (815054, 815068), (851359, 851372), (871485, 871500), (916369, 916383), (916375, 916389), (916426, 916439), (1135235, 1135249), (1135287, 1135300), (1139253, 1139266), (1139309, 1139322), (1139365, 1139378), (1139426, 1139441), (1139479, 1139492), (1275690, 1275703), (1275746, 1275761), (1275752, 1275767), (1652252, 1652267), (1652497, 1652512), (1667829, 1667844), (1667835, 1667850), (1667887, 1667900), (1687612, 1687628), (1687670, 1687683), (1687725, 1687738), (1773544, 1773559), (1775817, 1775833), (1775874, 1775888), (2007977, 2007990), (2008034, 2008047), (2018786, 2018802), (2057603, 2057619), (2063358, 2063371), (2067691, 2067706), (2074874, 2074887), (2153552, 2153568), (2153558, 2153574), (2153616, 2153630), (2153831, 2153847), (2153837, 2153853), (2232733, 2232750), (2315138, 2315155), (2315197, 2315210), (2349510, 2349523), (2790571, 2790584), (2790627, 2790640), (2827693, 2827706)

GCA_000470845.1_Staphylococcus_aureus_subsp._aureus_SA957 strain=SA957
Number of occurrences: 61

(183809, 183825), (183868, 183884), (294037, 294050), (369181, 369194), (421403, 421419), (616642, 616655), (769598, 769613), (769656, 769670), (779040, 779053), (779096, 779109), (779155, 779168), (810628, 810642), (810685, 810698), (815947, 815960), (825058, 825071), (825114, 825129), (825172, 825186), (861495, 861508), (881621, 881636), (926505, 926519), (926511, 926525), (926562, 926575), (1145445, 1145459), (1145497, 1145510), (1149463, 1149476), (1149519, 1149532), (1149575, 1149588), (1149636, 1149651), (1149689, 1149702), (1286709, 1286722), (1286765, 1286780), (1286771, 1286786), (1664883, 1664898), (1665128, 1665143), (1680519, 1680532), (1700244, 1700260), (1700302, 1700315), (1700357, 1700370), (1786172, 1786187), (1788445, 1788461), (1788502, 1788516), (1978794, 1978807), (1978851, 1978864), (1989603, 1989619), (1989660, 1989673), (1989716, 1989729), (2028532, 2028548), (2034288, 2034301), (2038622, 2038637), (2045805, 2045818), (2124484, 2124500), (2124490, 2124506), (2124548, 2124562), (2124763, 2124779), (2124769, 2124785), (2286131, 2286148), (2286190, 2286203), (2320504, 2320517), (2746184, 2746197), (2746240, 2746253), (2783306, 2783319)

GCA_000470865.1_Staphylococcus_aureus_subsp._aureus_SA40_strain=SA40
Number of occurrences: 61

(165288, 165304), (165347, 165363), (275516, 275529), (402828, 402844), (598343, 598356), (751215, 751230), (751273, 751287), (760658, 760671), (760714, 760727), (760773, 760786), (792240, 792254), (792297, 792310), (797559, 797572), (806670, 806683), (806726, 806741), (806784, 806798), (843105, 843118), (863232, 863247), (908116, 908130), (908122, 908136), (908173, 908186), (1127049, 1127063), (1127101, 1127114), (1131066, 1131079), (1131122, 1131135), (1131178, 1131191), (1131239, 1131254), (1131292, 1131305), (1268289, 1268302), (1268345, 1268360), (1268351, 1268366), (1604291, 1604306), (1604536, 1604551), (1619868, 1619883), (1619874, 1619889), (1619926, 1619939), (1639651, 1639667), (1639709, 1639722), (1639764, 1639777), (1725584, 1725599), (1727857, 1727873), (1727914, 1727928), (1917559, 1917572), (1917616, 1917629), (1928368, 1928384), (1928425, 1928438), (1928481, 1928494), (1967297, 1967313), (1973052, 1973065), (1977386, 1977401), (1984569, 1984582), (2063246, 2063262), (2063252, 2063268), (2063310, 2063324), (2063525, 2063541), (2063531, 2063547), (2142488, 2142505), (2224895, 2224912), (2224954, 2224967), (2259267, 2259280), (2722075, 2722088)

GCA_000237265.1_Staphylococcus_aureus_subsp._aureus_LGA251_strain=LGA251
Number of occurrences: 60

(24713, 24732), (24719, 24738), (73377, 73394), (73383, 73400), (172807, 172820), (172863, 172877), (172920, 172936), (221798, 221811), (287478, 287491), (287589, 287602), (361577, 361590), (361632, 361645), (361687, 361700), (517070, 517083), (632575, 632591), (757486, 757499), (757547, 757562), (803531, 803544), (812515, 812524), (847938, 847951), (847993, 848006), (848048, 848062), (848105, 848120), (865831, 865844), (954995, 955008), (955051, 955064), (955112, 955126), (1167652, 1167667), (1167705, 1167718), (1167766, 1167781), (1171684, 1171698), (1171742, 1171757), (1171794, 1171807), (1255722, 1255735), (1456791, 1456804), (1642852, 1642865), (1642966, 1642981), (1657559, 1657574), (1657565, 1657580), (1677227, 1677243), (1677285, 1677298), (1763631, 1763646), (1765849, 1765865), (1765963, 1765977), (1849897, 1849910), (1967835, 1967848), (1978342, 1978355), (1978398, 1978412), (2024493, 2024506), (2040162, 2040175), (2057557, 2057572), (2057563, 2057578), (2116598, 2116611), (2116657, 2116671), (2273450, 2273467), (2273509, 2273522), (2273565, 2273582), (2411393, 2411406), (2411449, 2411462), (2744602, 2744615)

### GCA_001880265.1_Staphylococcus_aureus_strain=SA40TW

Number of occurrences: 60

(163415, 163431), (163474, 163490), (273641, 273654), (400950, 400966), (601961, 601974), (754958, 754973), (755016, 755030), (764400, 764413), (764456, 764469), (764515, 764528), (795981, 795995), (796038, 796051), (801300, 801313), (810409, 810422), (810465, 810480), (810523, 810537), (847039, 847052), (867166, 867181), (912178, 912192), (912184, 912198), (912235, 912248), (1131239, 1131253), (1131291, 1131304), (1135256, 1135269), (1135312, 1135325), (1135368, 1135381), (1135429, 1135444), (1135482, 1135495), (1272447, 1272460), (1272503, 1272518), (1272509, 1272524), (1605629, 1605644), (1605874, 1605889), (1621206, 1621221), (1621212, 1621227), (1621264, 1621277), (1640989, 1641005), (1641047, 1641060), (1641102, 1641115), (1727050, 1727065), (1729322, 1729338), (1729379, 1729393), (1960128, 1960141), (1960185, 1960198), (1970937, 1970953), (1970994, 1971007), (2009810, 2009826), (2015565, 2015578), (2019898, 2019913), (2027081, 2027094), (2105754, 2105770), (2105760, 2105776), (2105818, 2105832), (2106033, 2106049), (2106039, 2106055), (2184994, 2185011), (2267396, 2267413), (2267455, 2267468), (2301768, 2301781), (2765617, 2765630)

GCA_000452385.2_Staphylococcus_aureus_subsp._aureus_Tager_104_strain=Tager_104
Number of occurrences: 57

(109783, 109796), (124043, 124058), (124049, 124064), (273423, 273438), (273429, 273444), (273481, 273494), (410880, 410895), (410938, 410951), (410994, 411007), (411050, 411065), (415018, 415031), (415074, 415088), (673895, 673908), (673951, 673965), (673957, 673971), (720556, 720571), (724692, 724705), (724747, 724762), (724753, 724768), (762684, 762699), (762742, 762757), (762798, 762812), (777039, 777052), (777100, 777114), (808565, 808578), (808621, 808634), (818003, 818017), (1155049, 1155062), (1172189, 1172205), (1315694, 1315707), (1315806, 1315819), (1426695, 1426711), (1426756, 1426770), (1569985, 1569998), (1880901, 1880914), (1880957, 1880971), (1880963, 1880977), (1909805, 1909817), (2014279, 2014292), (2048765, 2048778), (2048884, 2048901), (2224486, 2224499), (2300976, 2300989), (2308132, 2308147), (2308189, 2308205), (2312519, 2312532), (2362284, 2362297), (2401616, 2401629), (2453697, 2453712), (2528007, 2528020), (2612342, 2612356), (2612398, 2612414), (2614615, 2614629), (2614621, 2614635), (2700324, 2700340), (2700377, 2700390), (2734513, 2734527)

GCA_000210315.1_Staphylococcus_aureus_subsp._aureus_ED133_strain=ED133
Number of occurrences: 56

(43255, 43272), (43261, 43278), (142915, 142931), (258180, 258193), (554998, 555011), (555058, 555074), (637215, 637228), (678846, 678859), (787298, 787313), (788239, 788254), (834254, 834267), (843475, 843488), (843531, 843546), (882712, 882725), (927704, 927717), (927765, 927779), (1181517, 1181531), (1181569, 1181582), (1185549, 1185563), (1185601, 1185614), (1185663, 1185678), (1470093, 1470106), (1470148, 1470163), (1470154, 1470169), (1470263, 1470278), (1668599, 1668614), (1668605, 1668620), (1688210, 1688223), (1776586, 1776600), (1860410, 1860423), (2028456, 2028469), (2039027, 2039043), (2039084, 2039097), (2039140, 2039154), (2085236,2085249), (2092387, 2092402), (2092444, 2092459), (2115206, 2115219), (2132012, 2132025), (2132073, 2132088), (2191305, 2191318), (2273308, 2273321), (2273364, 2273377), (2355804, 2355821), (2355863, 2355880), (2355922, 2355939), (2355982, 2355999), (2371330, 2371345), (2371336, 2371351), (2390581, 2390594), (2390637, 2390650), (2495351, 2495363), (2495405, 2495418), (2524741, 2524754), (2796833, 2796846), (2826188, 2826201)

### GCA_900004855.1-Staphylococcus_aureus_strain=BB155

Number of occurrences: 54

(123123, 123138), (123181, 123194), (138528, 138540), (283355, 283368), (319928, 319941), (319984, 319997), (717339, 717352), (717395, 717411), (739206, 739222), (748695, 748708), (780097, 780111), (780153, 780166), (780209, 780220), (794274, 794289), (831374, 831387), (831429, 831442), (835828, 835841), (881424, 881437), (881480, 881493), (881536, 881549), (881597, 881612), (902396, 902412), (968221, 968234), (1096965, 1096978), (1097021, 1097034), (1165678, 1165692), (1165735, 1165748), (1165791, 1165804), (1618451, 1618466), (1618718, 1618731), (1618773, 1618786), (1652734,1652750), (1652792, 1652808), (1652850, 1652866), (1738654, 1738668), (1738710, 1738724), (1738766, 1738780), (1740931, 1740947), (1740989, 1741003), (1779245, 1779259), (1779301, 1779316), (1779359, 1779372), (1906868, 1906881), (1989513, 1989526), (1989740, 1989755), (1991308, 1991321), (1991364, 1991377), (2000630, 2000645), (2076904, 2076919), (2076961, 2076976), (2178945, 2178960), (2445262, 2445275), (2689495, 2689508), (2689550, 2689565)

### GCA_001456215.1_Staphylococcus_aureus_strain=MS4

Number of occurrences: 53

(183699, 183715), (183758, 183774), (293927, 293940), (421237, 421253), (774585, 774600), (774643, 774657), (784028, 784041), (784084, 784097), (784143, 784156), (815616, 815630), (815673, 815686), (820935, 820948), (830046, 830059), (830102, 830117), (830160, 830174), (866627, 866640), (886753, 886768), (931637, 931651), (931643, 931657), (931694, 931707), (1150570, 1150584), (1150622, 1150635), (1154588, 1154601), (1154644, 1154657), (1154700, 1154713), (1154761, 1154776), (1154814, 1154827), (1292047, 1292060), (1292103, 1292118), (1292109, 1292124), (1712339, 1712354), (1712584, 1712599), (1727916, 1727931), (1727922, 1727937), (1727974, 1727987), (1747699, 1747715), (1747757, 1747770), (1747812, 1747825), (1833764, 1833779), (1836037, 1836053), (1836094, 1836108), (2041015, 2041031), (2041021, 2041037), (2041079, 2041093), (2041294, 2041310), (2041300, 2041316), (2120257, 2120274), (2202662, 2202679), (2202721, 2202734), (2237034, 2237047), (2666117, 2666130), (2666173, 2666186), (2703239, 2703252)

On each Pseudomonas genome where nucleotide repeat elements RPSEUDO occur, 10 flanking genes both upstream and downstream were found on each strand (+and −) of DNA. Similarly for Staphylococcus genome where nucleotide repeat elements STAR and RSTAPH occur, 10 flanking genes both upstream and downstream were found on each strand (+and −) of DNA. Functional annotation of these genes was performed using HMM search with PFAM as the database. Functional categorization of these genes on the basis of pathways they are involved in was carried out using literature mining. The broad categories have been discussed in Tables 1 and 2.

Following is the number of occurrences and locations of R-PSEUDO repeats in the strains from Pseudomonas aeruginosa is as follows. Due the large number of available strain, only top and well characterized few are provided below:

### Pseudomonas_aeruginosa_PAO1_-_GCA_000006765.1_ASM676v1

Number of occurrences: 101

[(264567, 264596), (264614, 264642), (264668, 264697), (264715, 264743), (264769, 264798), (264816, 264844), (264870, 264899), (501230, 501259), (501274, 501303), (521332, 521361), (521408, 521437), (521453, 521482), (521529, 521558), (521570, 521599), (529976, 530005), (570929, 570958), (570988, 571016), (849153, 849181), (865589, 865618), (950796, 950825), (950853, 950882), (1248677, 1248706), (1248982, 1249011), (1447113, 1447142), (1474133, 1474162), (1495997, 1496026), (1749270, 1749298), (1882504, 1882533), (2076983, 2077011), (2136408, 2136437), (2189974, 2190003), (2199728, 2199756), (2250710, 2250739), (2486118, 2486146), (2486165, 2486194), (2486248, 2486276), (2486295, 2486324), (2556734, 2556762), (2558369, 2558397), (2558500, 2558528), (2558631, 2558659), (2558763, 2558791), (2705345, 2705373), (2705389, 2705418), (2705460, 2705488), (2799814, 2799843), (3618663, 3618692), (3841768, 3841798), (3841824, 3841853), (3841870, 3841899), (3841925, 3841954), (3843398, 3843427), (3843444, 3843473), (3843499, 3843528), (3843545, 3843574), (3843600, 3843629), (3847558, 3847586), (3858404, 3858433), (3873962, 3873991), (3874033, 3874061), (3874077, 3874106), (3874192, 3874221), (3874307, 3874336), (3874537, 3874566), (3874608, 3874636), (3988712, 3988740), (3988756, 3988785), (4008842, 4008870), (4045889, 4045918), (4214588, 4214617), (4376810, 4376838), (4377024, 4377053), (4377069, 4377097), (4377198, 4377226), (4403460, 4403489), (4528449, 4528478), (4528498, 4528527), (4528611, 4528640), (4588625, 4588653), (4595744, 4595773), (4672734, 4672763), (4672819, 4672848), (4672931, 4672960), (4699884, 4699913), (4705453, 4705482), (4705498, 4705526), (4720088, 4720116), (4858422, 4858451), (5017908, 5017937), (5050741, 5050770), (5361258, 5361286), (5372337, 5372366), (5455125, 5455153), (5455182, 5455211), (5455231, 5455259), (5471508, 5471536), (5774948, 5774977), (5774993, 5775022), (5775093, 5775122), (5779707, 5779736), (6222889, 6222918)] Pseudomonas_aeruginosa_RP73_-_GCA_000414035.1_ASM41403v1

Number of occurrences: 102

[(258734, 258763), (494421, 494450), (514211, 514240), (514287, 514316), (514332, 514361), (514408, 514437), (514453, 514482), (522860, 522889), (562732, 562761), (562791, 562819), (562861, 562890), (562920, 562948), (814698, 814726), (831134, 831163), (913988, 914017), (1309597, 1309626), (1547104, 1547133), (1808306, 1808334), (1914507, 1914536), (1914628, 1914657), (2109125, 2109153), (2109222, 2109251), (2109269, 2109297), (2109366, 2109395), (2164534, 2164563), (2218110, 2218139), (2227862, 2227890), (2276108, 2276137), (2334459, 2334487), (2334503, 2334532), (2530744, 2530772), (2530791, 2530820), (2530874, 2530902), (2530921, 2530950), (2601352, 2601380), (2601483, 2601511), (2601615, 2601643), (2749081, 2749109), (2749125, 2749154), (2749195, 2749223), (2749239, 2749268), (2749309, 2749337), (2749424, 2749452), (2774826, 2774854), (2806134, 2806163), (2806179, 2806208), (2825142, 2825171), (3119172, 3119201), (3701210, 3701239), (3924314, 3924343), (3924360, 3924389), (3924415, 3924444), (3924461, 3924490), (3924516, 3924545), (3924562, 3924591), (3924617, 3924646), (3924663, 3924692), (3924718, 3924747), (3939522, 3939551), (3955037, 3955065), (4045444, 4045473), (4065557, 4065585), (4102483, 4102512), (4271179, 4271208), (4443822, 4443850), (4443951, 4443979), (4444035, 4444064), (4444080, 4444108), (4444165, 4444194), (4444210, 4444238), (4470470, 4470499), (4470539, 4470568), (4540539, 4540568), (4594072, 4594101), (4594121, 4594150), (4654133, 4654161), (4661252, 4661281), (4738261, 4738290), (4738346, 4738375), (4765315, 4765344), (4770884, 4770913), (4770929, 4770957), (4785531, 4785559), (4785628, 4785657), (4923849, 4923878), (4923915, 4923944), (4923960, 4923989), (5083390, 5083419), (5116230, 5116259), (5116343, 5116372), (5425979, 5426007), (5437262, 5437291), (5447417, 5447446), (5525927, 5525955), (5542310, 5542338), (5843974, 5844003), (5844019, 5844048), (5844119, 5844148), (5848733, 5848762), (5848778, 5848807), (6300506, 6300535), (6302300, 6302328)]

### Pseudomonas_aeruginosa_PA1_-_GCA_000496605.2_ASM49660v2

Number of occurrences: 98 [(271651, 271680), (271698, 271726), (271752, 271781), (271799, 271827), (271854, 271883), (497253, 497282), (497310, 497339), (497354, 497383), (517152, 517181), (525559, 525588), (565425, 565454), (565484, 565512), (565555, 565584), (565614, 565642), (565684, 565713), (565743, 565771), (792452, 792481), (807094, 807122), (812707, 812736), (839676, 839705), (839788, 839817), (839900, 839929), (839985, 840014), (916962, 916991), (924082, 924110), (983603, 983632), (983652, 983681), (1037188, 1037217), (1107197, 1107226), (1107266, 1107295), (1133549, 1133577), (1545402, 1545431), (1582458, 1582486), (1602543, 1602572), (1602588, 1602616), (1693018, 1693046), (1693088, 1693117), (1693203, 1693232), (1693248, 1693276), (1693318, 1693347), (1693433, 1693462), (1702250, 1702279), (1709061, 1709090), (1719908, 1719936), (1723865, 1723894), (1723919, 1723948), (1723965, 1723994), (1724020, 1724049), (1724066, 1724095), (1724121, 1724151), (2785380, 2785409), (2866108, 2866136), (2866178, 2866207), (2866223, 2866251), (2866338, 2866366), (2866452, 2866480), (3013315, 3013343), (3083754, 3083783), (3083802, 3083830), (3274555, 3274584), (3274600, 3274628), (3335133, 3335162), (3335258, 3335287), (3335383, 3335412), (3335507, 3335536), (3335631, 3335660), (3396120, 3396149), (3449687, 3449716), (3542636, 3542664), (3542682, 3542711), (3737964, 3737993), (3871276, 3871304), (4093909, 4093938), (4109860, 4109889), (4730551, 4730580), (4730608, 4730637), (4874166, 4874195), (4890603, 4890631), (5070020, 5070049), (5070065, 5070094), (5070131, 5070160), (5070176, 5070205), (5070241, 5070270), (5070286, 5070315), (5262705, 5262734), (5573227, 5573255), (5573429, 5573457), (5584297, 5584326), (5596100, 5596129), (5606219, 5606248), (5677394, 5677422), (5693671, 5693699), (6000339, 6000368), (6000384, 6000413), (6000483, 6000512), (6005097, 6005126), (6449386, 6449415), (6451180, 6451208)]

### Pseudomonas_aeruginosa_M18_-_GCA_000226155.1_ASM22615v1

Number of occurrences: 102

[(256823, 256852), (256924, 256953), (257025, 257054), (257072, 257100), (257126, 257155), (493325, 493354), (513463, 513492), (513508, 513537), (513584, 513613), (513629, 513658), (513705, 513734), (522112, 522141), (563071, 563100), (563130, 563158), (789051, 789079), (803653, 803681), (803697, 803726), (809266, 809295), (836245, 836274), (836357, 836386), (836469, 836498), (836554, 836583), (913544, 913573), (920664, 920692), (980643, 980672), (980692, 980721), (1034228, 1034257), (1104309, 1104338), (1130575, 1130603), (1293309, 1293338), (1504694, 1504723), (1543502, 1543530), (1563590, 1563619), (1563635, 1563663), (1654050, 1654078), (1654120, 1654149), (1654165, 1654193), (1654235, 1654264), (1654351, 1654380), (1669908, 1669937), (1680755, 1680783), (1684712, 1684741), (1684766, 1684795), (1684812, 1684841), (1909243, 1909272), (2799119, 2799148), (2818082, 2818111), (2818127, 2818156), (2852867, 2852895), (2889120, 2889148), (2889190, 2889219), (2889235, 2889263), (3034314, 3034342), (3034445, 3034473), (3104890, 3104919), (3104938, 3104966), (3105020, 3105049), (3105068, 3105096), (3334909, 3334938), (3385896, 3385924), (3395647, 3395676), (3449224, 3449253), (3504054, 3504082), (3504198, 3504226), (3504244, 3504273), (3504486, 3504514), (3697249, 3697278), (3697370, 3697399), (3943760, 3943789), (4310235, 4310264), (4310540, 4310569), (4607303, 4607332), (4607360, 4607389), (4690057, 4690086), (4706494, 4706522), (4897423, 4897452), (4897489, 4897518), (4897534, 4897563), (4897600, 4897629), (4897645, 4897674), (4897711, 4897740), (4897756, 4897785), (4897822, 4897851), (4897867, 4897896), (4897933, 4897962), (4897978, 4898007), (4898044, 4898073), (4898089, 4898118), (5057529, 5057558), (5090481, 5090510), (5414153, 5414181), (5425131, 5425160), (5435250, 5435279), (5506420, 5506448), (5506477, 5506506), (5522697, 5522725), (5828406, 5828435), (5828451, 5828480), (5828550, 5828579), (5833164, 5833193), (6279067, 6279096), (6280861, 6280889)]

### Pseudomonas_aeruginosa_DK1_-_GCA_900069025.1_ASM90006902v1

Number of occurrences: 94 [(269615, 269644), (269716, 269745), (505907, 505936), (505951, 505980), (525818, 525847), (534270, 534299), (575237, 575266), (575296, 575324), (792915, 792943), (807517, 807545), (807561, 807590), (813130, 813159), (840093, 840122), (840178, 840207), (917188, 917217), (924296, 924324), (984318, 984347), (984367, 984396), (1037899, 1037928), (1119286, 1119315), (1119355, 1119384), (1145655, 1145683), (1145781, 1145809), (1145825, 1145854), (1145910, 1145938), (1145954, 1145983), (1146169, 1146197), (1309907, 1309936), (1478593, 1478622), (1538384, 1538413), (1628813, 1628841), (1628883, 1628912), (1628998, 1629027), (1644552, 1644581), (1659357, 1659386), (1659412, 1659441), (1659458, 1659487), (1659513, 1659543), (1886602, 1886631), (2473051, 2473080), (2716692, 2716721), (2769863, 2769891), (2806595, 2806623), (2806710, 2806738), (2806824, 2806852), (2953850, 2953878), (2953943, 2953971), (3024318, 3024347), (3024366, 3024394), (3024445, 3024474), (3024493, 3024521), (3186638, 3186667), (3186683, 3186711), (3247337, 3247366), (3305351, 3305380), (3358762, 3358791), (3411145, 3411173), (3411191, 3411220), (3605175, 3605204), (3605296, 3605325), (3973792, 3973821), (3987694, 3987723), (4014707, 4014736), (4515500, 4515529), (4515557, 4515586), (4609605, 4609634), (4626047, 4626075), (4805976, 4806005), (4806042, 4806071), (4806087, 4806116), (4806153, 4806182), (4806198, 4806227), (4806264, 4806293), (4806309, 4806338), (4806375, 4806404), (4806420, 4806449), (4998294, 4998323), (4998633, 4998662), (5309422, 5309450), (5309523, 5309551), (5320501, 5320530), (5330657, 5330686), (5401880, 5401909), (5401929, 5401957), (5401986, 5402015), (5402035, 5402063), (5418317, 5418345), (5721735, 5721764), (5721780, 5721809), (5721880, 5721909), (5726494, 5726523), (5726539, 5726568), (6171012, 6171041), (6172806, 6172834)]

In the present example, the RPSEUDO, STAR element and RSTAPH sequences are palindromic and may form a hairpin loop structure indicating their role in regulation of transcription. These loops may either form at DNA level or at the ends of their mRNA during DNA transcription. This hairpin loop in the mRNA could be involved in prevention of the early decay of mRNA, resulting in higher protein formation of the virulence genes which are in the vicinity of these palindromic elements. Reduction in pathogenicity can be achieved by decreasing the stability of mRNA corresponding to these virulent genes which can be attained by removing the hairpin loops. If hairpin loop formation takes place at DNA level it might regulate DNA supercoiling and concatenation. The hairpin loop is not followed by a polyA tail indicating it might not be working as transcription terminator.

Depending on the presence of the hairpin loop structure, one of the strategies mentioned above can be used to combat infections due to Pseudomonas aeruginosa and Staphylococcus aureus.

The embodiments of present disclosure herein provides a method and system for combating infections due to Pseudomonas aeruginosa and Staphylococcus aureus.

Sequences and their reverse complements have been disclosed

Sequence 001:
Pseudomonas aeruginosa:
GGCGNATAACNNCN(₂₋₄)GNNGTTATNCGCC
Sequence 002:
Staphylococcus aureus:
GTTG(N)₀₋₅(GC)₀₋₆(N)₀₋₅CAAC
Sequence 003:
Staphylococcus aureus:
GGTGGGACGACGAAATAAATTTTGCGAAAATATCATTTCTGTCCCACT
CCCAA

where N refers to any nucleotide out of A, T, G and C and numeric values in subscript indicate the range of the number of times a nucleotide or a set of nucleotides is repeated in the sequence.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments of present disclosure herein address unresolved problem of hospital acquired infections (HAIs) which are notoriously difficult to treat as the HAI agents develop resistance to most form of antibiotics. The embodiment provides a system and method for combating infections due to Pseudomonas aeruginosa and Staphylococcus aureus.

It is to be understood that the scope of the protection is extended to such a program and in addition to a computer-readable means having a message therein; such computer-readable storage means contain program-code means for implementation of one or more steps of the method, when the program runs on a server or mobile device or any suitable programmable device. The hardware device can be any kind of device which can be programmed including e.g. any kind of computer like a server or a personal computer, or the like, or any combination thereof. The device may also include means which could be e.g. hardware means like e.g. an application-specific integrated circuit (ASIC), a field-programmable gate array (FPGA), or a combination of hardware and software means, e.g. an ASIC and an FPGA, or at least one microprocessor and at least one memory with software processing components located therein. Thus, the means can include both hardware means and software means. The method embodiments described herein could be implemented in hardware and software. The device may also include software means. Alternatively, the embodiments may be implemented on different hardware devices, e.g. using a plurality of CPUs.

The embodiments herein can comprise hardware and software elements. The embodiments that are implemented in software include but are not limited to, firmware, resident software, microcode, etc. The functions performed by various components described herein may be implemented in other components or combinations of other components. For the purposes of this description, a computer-usable or computer readable medium can be any apparatus that can comprise, store, communicate, propagate, or transport the program for use by or in connection with the instruction execution system, apparatus, or device.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope of the disclosed embodiments. Also, the words “comprising,” “having,” “containing,” and “including,” and other similar forms are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, nonvolatile memory, hard drives, CD ROMs, DVDs, flash drives, disks, and any other known physical storage media.

It is intended that the disclosure and examples be considered as exemplary only, with a true scope of disclosed embodiments being indicated by the following claims.

What is claimed is:

1. A method for combating infections due to Pseudomonas aeruginosa and Staphylococcus aureus, the method comprising:
obtaining a sample from an infected area;
isolating and extracting DNA from the obtained sample using one of a laboratory method;
sequencing the isolated DNA using a sequencer;
identifying a first set of nucleotide repeat sequences in the sequenced DNA which are occurring more than a predefined number of times in Pseudomonas aeruginosa;
identifying a second set of nucleotide repeat sequences in the extracted DNA which are occurring more than a predefined number of times in Staphylococcus aureus;
identifying a first set of neighborhood genes present upstream and downstream of the first set of nucleotide repeat sequences;
identifying a second set of neighborhood genes present upstream and downstream of the second set of nucleotide repeat sequences;
annotating the first and second set of neighborhood genes according to their functional roles in their respective pathogen based on their involvement in pathways in the identified set of neighborhood genes; and
testing the presence of a secondary structure in the identified first and second set of nucleotide repeat sequences;
preparing and administering an engineered polynucleotide construct on the infected area depending on the presence of the secondary structure to combat the infections due to Pseudomonas aeruginosa and Staphylococcus aureus, wherein the engineered polynucleotide construct is comprising:
one or more of the first and the second set of nucleotide rep eat sequences with multiple copies at dispersed locations on the candidate pathogen genomes of one or more of the Pseudomonas or Staphylococcus, wherein the first set of nucleotide repeat sequences comprises a Sequence ID 001 or complement of the sequence ID 001, and the second set of nucleotide repeat sequences comprises one or more of a Sequence ID 002, a Sequence ID 003, complement of the Sequence ID 002 or complement of the Sequence ID 003,
a first enzyme capable of nicking and cleaving the identified set of nucleotide sequences, and
a second enzyme capable of removal of a set of neighborhood genes flanking the set of nucleotide repeat sequences;
checking the efficacy of the administered engineered polynucleotide construct to combat the infections due to Pseudomonas aeruginosa and Staphylococcus aureus after a predefined time period; and
re-administering the engineered polynucleotide construct if Pseudomonas aeruginosa and Staphylococcus aureus are still present in the infected area post administering.

2. The method according to claim 1 wherein the samples obtained from infected area is one or more of fecal matter, blood, urine, tissue biopsy, hospital surfaces or environmental samples.

3. The method according to claim 1 wherein the DNA isolation and extraction methods may comprise of laboratory standardized protocols including DNA isolation and extraction kits.

4. The method according to claim 1 wherein the plurality of pathogen detection method comprises one or more of:
a sequencing technique,
a flow cytometry based methodology,
a microscopic examination of the microbes in collected sample,
a microbial culture of pathogens in vitro, immunoassays, cell toxicity assay, enzymatic, colorimetric or fluorescence assays, assays involving spectroscopic/spectrometric/chromatographic identification and screening of signals from complex microbial populations.

5. The method according to claim 1, wherein the pathogen detection may also comprise of one or more of sequenced microbial DNA data, a microscopic imaging data, a flow cytometry cellular measurement data, a colony count and cellular phenotypic data of microbes grown in in-vitro cultures, immunological data, proteomic/metabolomics data, and a signal intensity data.

6. The method according to claim 1 further comprising sequenced microbial data, wherein the sequenced microbial data comprises sequences obtained from sequencing platforms comprising sequences of marker genes including 16S rRNA, Whole Genome Shotgun (WGS) sequences, sequences obtained from a fragment library, sequences from a mate-pair library or a paired-end library based sequencing technique, a complete sequence of pathogen genome or a combination thereof, wherein, the pathogen detection in the sample may depend on identification of taxonomic groups from these sequences.

7. The method according to claim 1, wherein the polynucleotides are inserted into vectors which allow insertion of external DNA fragments, wherein the engineered polynucleotide construct is carried by plasmid or phage based cloning vectors, wherein the engineered polynucleotide construct further comprise of bacteria specific promoter sequence, a terminator sequence, a stretch of Thymine nucleotides which is transcribed into a polyA tail for stabilizing the mRNAs transcripts corresponding to each enzyme, wherein the promoters and terminators specific to candidate bacteria can be utilized in the engineered polynucleotide construct.

8. The method according to claim 1 wherein the engineered polynucleotide construct comprises of a CRISPR-Cas system, comprising:
a CRISPR enzyme,
a guide sequence capable of hybridizing to the identified target nucleotide repeat sequence within the pathogen genome,
a tracr mate sequence, and
a tracr sequence,
wherein the guide sequence, the tracr mate and the tracr sequences are linked to one regulatory element of the engineered polynucleotide construct while the CRISPR enzyme is linked to another regulatory module within the vector.

9. The method according to claim 1, wherein the engineered polynucleotide construct is administered using one or more of following delivery methods:
liposome encompassing the engineered polynucleotide construct,
targeted liposome with a ligand specific to the target pathogen on the external surface and encompassing the engineered polynucleotide construct to be administered,
using nanoparticles like Ag and Au,
gene guns or micro-projectiles where the engineered polynucleotide construct is adsorbed or covalently linked to heavy metals which carry it to different bacterial cells, or
bacterial conjugation methods and bacteriophage specific to the targeted pathogen.

10. The method according to claim 1, wherein the first enzyme is a nicking enzyme and the second enzyme is a cleaving enzyme.

11. The method according to claim 1, wherein the first and the second set of nucleotide repeat sequences corresponding to one or more than one strain of the Pseudomonas aeruginosa and Staphylococcus aureus or candidate genus or species, wherein the first and the second set of nucleotide repeat sequences are found in multiple copies at distant locations on the genomes of all pathogenic strains of candidate genus or specie and these nucleotide repeat sequences do not show more than two nucleotide sequence similarity based match to genome sequences corresponding to genera or species other than the genome sequences of pathogens belonging to the candidate genus or species or with genomes of commensal strains within the candidate genus or specie; wherein distant locations refer to distance of greater than 10000 nucleotide base pairs.

12. The method according to claim 1 further comprising the step identifying the first and the second set of nucleotide repeat sequences comprises:
selecting a nucleotide sequence stretches of a predefined length Rₙ from the genomes of strains of candidate pathogen with a difference in the start position of two consecutive nucleotide stretches Rₙᵢ₊₁ and Rₙᵢ as 5 nucleotides, wherein the predefined length refers to the length of a stretch of nucleotide sequence picked from the complete nucleotide sequence of a bacterial genome, used as a seed input for local sequence alignment tools,
aligning a stretch of sequences within the genome of candidate pathogen genus/specie or with genomes of all strains of the candidate pathogen genus/specie Pseudomonas aeruginosa and Staphylococcus aureus, and
identifying the first and second set of nucleotide repeat sequences, repeating more than 10 times at distant locations on the bacterial genome as the set of nucleotide repeat sequences, wherein the first set of nucleotide repeat sequences comprises a Sequence ID 001 or complement of the sequence ID 001, and the second set of nucleotide repeat sequences comprises one or more of a Sequence ID 002, a Sequence ID 003, complement of the Sequence ID 002 or complement of the Sequence ID 003.

13. The method according to claim 1, wherein the first and the second set of nucleotide repeat sequences are in genomic neighborhood of or flanking the genes encoding proteins with essential functions within a pathogen genome, wherein the genomic neighborhood refers to regions lying within a predefined number of genes to the selected nucleotide repeat sequence or the reverse complement of the selected nucleotide repeat sequence on the candidate pathogen genome or lying within a distance of predefined number of bases with respect to the selected nucleotide repeat sequence on the genome of the pathogen wherein, the important functional genes refer to the genes in pathogens which encode for proteins which are critical for survival, pathogenicity, interaction with the host, adherence to the host or for the virulence of bacteria, wherein the minimum predefined number of genes to be considered in genomic neighborhood is 10.

14. The method according to claim 1, wherein the non-culturable taxonomic groups or pathogens within a sample collected from an environment is obtained by amplification of marker genes like 16S rRNA within bacteria.

15. The method according to claim 1, wherein the information and detection of non-culturable taxonomic groups or pathogens within a sample is obtained by the binning of whole genome sequencing reads into various taxonomic groups using different methods including sequence similarities as well as several methods using supervised and unsupervised classifiers for taxonomic binning of metagenomics sequences.

16. The method according to claim 1, wherein the distant locations may refer to distance of greater than 10000 nucleotide base pairs, and wherein the sequence matching is performed by processor implemented tools for nucleotide sequence alignment which may comprise PILER, BLAST or Burrows wheeler alignment tool.

17. The method according to claim 1, wherein the pathogens is identified by amplification of marker genes like 16S rRNA and obtaining their abundance.

18. The method according to claim 1, wherein the taxonomic constitution of the sample is obtained from these 16S rRNA sequences using standardized methodologies, wherein the taxonomic constitution is utilized to determine occurrence of pathogens in the samples.

19. A system for combating infections due to Pseudomonas aeruginosa and Staphylococcus aureus, the system comprises:
a sample collection module for obtaining a sample from an infected area;
a pathogen detection and DNA extraction module isolating DNA from the obtained sample using one of a laboratory methods;
a sequencer for sequencing the isolated DNA;
one or more hardware processors;
a memory in communication with the one or more hardware processors, wherein the one or more first hardware processors are configured to execute programmed instructions stored in the one or more first memories, to:
identify a first set of nucleotide repeat sequences in the sequenced DNA which are occurring more than a predefined number of times in Pseudomonas aeruginosa;
identify a second set of nucleotide repeat sequences in the extracted DNA which are occurring more than a predefined number of times in Staphylococcus aureus;
identify a first set of neighborhood genes present upstream and downstream of the first set of nucleotide repeat sequences;
identify a second set of neighborhood genes present upstream and downstream of the second set of nucleotide repeat sequences;
annotate the first and second set of neighborhood genes according to their functional roles in their respective pathogen based on their involvement in pathways in the identified set of neighborhood genes; and
test the presence of a secondary structure in the identified first and second set of nucleotide repeat sequences;
an administration module configured to prepare and administer an engineered polynucleotide construct on the infected area depending on the presence of the secondary structure to combat the infections due to Pseudomonas aeruginosa and Staphylococcus aureus, wherein the engineered polynucleotide construct is comprising:
one or more of the first and the second set of nucleotide repeat sequences with multiple copies at dispersed locations on the candidate pathogen genomes of one or more of the Pseudomonas or Staphylococcus, wherein the first set of nucleotide repeat sequences comprises a Sequence ID 001 or complement of the sequence ID 001, and the second set of nucleotide repeat sequences comprises one or more of a Sequence ID 002, a Sequence ID 003, complement of the Sequence ID 002 or complement of the Sequence ID 003,
a first enzyme capable of nicking and cleaving the identified set of nucleotide sequences, and
a second enzyme capable of removal of a set of neighborhood genes flanking the set of nucleotide repeat sequences; and
an efficacy module configured to
check the efficacy of the administered engineered polynucleotide construct to combat the infections due to Pseudomonas aeruginosa and Staphylococcus aureus after a predefined time period; and
re-administering the engineered polynucleotide construct if the Pseudomonas aeruginosa and Staphylococcus aureus are still present in the infected area post administering.

20. One or more non-transitory machine readable information storage mediums comprising one or more instructions which when executed by one or more hardware processors cause:
obtaining a sample from an infected area;
isolating and extracting DNA from the obtained sample using one of a laboratory method;
sequencing the isolated DNA using a sequencer;
identifying a first set of nucleotide repeat sequences in the sequenced DNA which are occurring more than a predefined number of times in Pseudomonas aeruginosa;
identifying a second set of nucleotide repeat sequences in the extracted DNA which are occurring more than a predefined number of times in Staphylococcus aureus;
identifying a first set of neighborhood genes present upstream and downstream of the first set of nucleotide repeat sequences;
identifying a second set of neighborhood genes present upstream and downstream of the second set of nucleotide repeat sequences;
annotating the first and second set of neighborhood genes according to their functional roles in their respective pathogen based on their involvement in pathways in the identified set of neighborhood genes; and
testing the presence of a secondary structure in the identified first and second set of nucleotide repeat sequences;
preparing and administering an engineered polynucleotide construct on the infected area depending on the presence of the secondary structure to combat the infections due to Pseudomonas aeruginosa and Staphylococcus aureus, wherein the engineered polynucleotide construct is comprising:
one or more of the first and the second set of nucleotide repeat sequences with multiple copies at dispersed locations on the candidate pathogen genomes of one or more of the Pseudomonas or Staphylococcus, wherein the first set of nucleotide repeat sequences comprises a Sequence ID 001 or complement of the sequence ID 001, and the second set of nucleotide repeat sequences comprises one or more of a Sequence ID 002, a Sequence ID 003, complement of the Sequence ID 002 or complement of the Sequence ID 003,
a first enzyme capable of nicking and cleaving the identified set of nucleotide sequences, and
a second enzyme capable of removal of a set of neighborhood genes flanking the set of nucleotide repeat sequences;
checking the efficacy of the administered engineered polynucleotide construct to combat the infections due to Pseudomonas aeruginosa and Staphylococcus aureus after a predefined time period; and
re-administering the engineered polynucleotide construct if Pseudomonas aeruginosa and Staphylococcus aureus are still present in the infected area post administering.
